(19) United States
(12) Patent Application Publication
(10) Pub. No.: US 2017/0290853 A1
(43) Pub. Date: Oct. 12, 2017

(54) METHODS TO FACILITATE THE SOLUBILIZATION OF BETA-1,3-GLUCAN AND ENHANCE IMMUNE FUNCTION AND OTHER RELATED USES

(71) Applicant: KEMIN INDUSTRIES, INC., Des Moines, IA (US)

(72) Inventors: Geoffrey Paul HORST, Grosse Pointe Farms, MI (US); Robert Bernard LEVINE, Ann Arbor, MI (US)

(21) Appl. No.: 15/486,093

(22) Filed: Apr. 12, 2017

Related U.S. Application Data
(60) Provisional application No. 62/321,603, filed on Apr. 12, 2016.

(51) Int. Cl. A61K 31/716; A61K 9/00; A61K 9/06; A61K 45/06

(52) U.S. Cl. CPC A61K 31/716; A61K 45/06; A61K 9/0053; A61K 9/0019; A61K 9/0014; A61K 9/06

(57) ABSTRACT

The present application relates to methods to facilitate the solubilization of beta-1,3-glucan in solution, for instance an aqueous or organic solvent and uses thereof to enhance immune function in an individual. Others aspects of the present invention relate to administering compositions or mixtures comprising solubilized beta-1,3-glucan to enhance immune function in humans or animals.

This application claims priority to U.S. Patent Application 61/321,603, filed Apr. 12, 2016, which is incorporated herein in its entirety by this reference.

### FIELD OF THE INVENTION

The present application relates to methods to facilitate the solubilization of beta-1,3-glucan in solution, for instance an aqueous or organic solvent and uses thereof to enhance immune function in an individual. Others aspects of the present invention relate to administering compositions or mixtures comprising solubilized beta-1,3-glucan to enhance immune function in humans or animals.

### BACKGROUND OF THE INVENTION

Beta glucans are polymers of D-glucose linked by beta-glycosidic bonds produced by a variety of organisms including yeast, fungi, bacteria, algae, oats, barley, and kelp. Different organisms produce beta glucans with differing branching structures, average molecular weights, solubility, and/or tertiary structure. For example, beta glucan derived from yeast is generally insoluble and has both beta-1,3 and 1,6-glycosidic bonds (beta-1,3-/1,6-glucan). On the other hand, beta glucan derived from oats is typically more soluble and has both 1,3- and 1,4-glycosidic bonds (beta-1,3-/1,4-glucan). In contrast, beta glucan derived from algae such as Euglena has almost exclusively 1,3-glycosidic bonds and no 1,6-glycosidic bonds. The specific glycosidic linkages of the various beta glucan forms affect the properties of these molecules.

Some beta glucans have been identified as having beneficial health properties. As beta glucan is typically associated with the surface of pathogenic microorganisms, the immune system of higher organisms has evolved to recognize beta glucan and to mount an immune response. For example, it has been shown that beta glucan derived from yeast can impact immune function by binding complement receptor 3 or dectin-1 on macrophages (see Brown et al., Journal of Experimental Medicine, vol. 196(3), pp. 407-412 (2002)). At the physiological level, beta glucan interacts with cell surface receptors to initiate a cascade of events including phagocytosis and the production of certain cytokines. By introducing certain beta glucans, the immune system can be primed so that its response to an actual disease challenge is more robust.

Modulation of the immune function in an individual to combat disease represents an alternative to the administration of conventional medicines. A modulated immune function may effectively treat a disease in an individual, or may prevent the onset of disease in an individual. Many conventional medicines cause undesirable side effects in patients. Furthermore, antibiotic-resistant strains of bacteria pose an ever-increasing health risk. As such, there is a need for alternative disease treatment that has fewer, if any, side effects. There is also a need for more natural methods to prevent the onset of disease.

Whereas beta glucans derived from yeast and oats have been extensively studied, the health benefits arising from beta-1,3-glucan derived from algae, such as Euglena, have received less attention. Moreover, previous studies performed on beta-1,3-glucan used beta-1,3-glucan in a water insoluble form known as particulate beta-1,3-glucan, which requires higher levels of beta-1,3-glucan than described herein to produce the desired effects.

Described herein are methods of enhancing immune function in an individual by administering low amounts of solubilized beta-1,3-glucan derived from Euglena. Certain diseases can be treated and/or prevented by enhancing the immune function of an individual by administering solubilized beta-1,3-glucan.

### SUMMARY OF THE INVENTION

Solubilized Euglena-derived beta-1,3-glucan is a potent immunomodulator. For example, even at low doses, solubilized Euglena-derived beta-1,3-glucan can cause cytokine production and increased expression of markers of immune cell activation. Accordingly, this application discloses a method of enhancing the immune function in an individual including administering to the individual an effective amount of a composition comprising solubilized Euglena-derived beta-1,3-glucan. In some variations, the Euglena-derived beta-1,3-glucan may be solubilized in a solution with a base.

In some variations, the effective amount of the composition is between 0.01 mg beta-1,3-glucan/kg body weight and 100 mg beta-1,3-glucan/kg body weight.

10101 In some aspects, solubilized Euglena-derived beta-1,3-glucan is more bioactive than a particulate form of beta-1,3-glucan derived from Euglena.

In some variations, administration of the composition comprising solubilized Euglena-derived beta-1,3-glucan modulates an autoimmune response, blood sugar level, cholesterol level, an infection, or inflammation. In some of these variations, inflammation is associated with allergies or intestinal inflammation. In other variations, the autoimmune response is associated with diabetes. In yet other variations, the infection is a bacterial, fungal, or viral infection.

In some variations, the Euglena can be heterotrophically grown. In some variations, the beta-1,3-glucan comprises paramylon. In some variations, the beta-1,3-glucan does not contain beta-1,6-glycosidic bonds.

In some variations, the composition may be administered daily as a single dose. In other variations, the composition may be administered as multiple separate doses in a single day.

In some variations, the composition may include an additional component such as alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene and combinations thereof.

In some variations, the composition may be administered orally. In some of these variations, the composition is added to drinking water. In other variations, the composition is administered intravenously. In yet other variations, the composition may be administered topically.

In some variations, the composition has a pH of greater than 7. In other variations the composition has a pH of less than 7. In still other variations, the composition has a pH of approximately 7.

In some variations, the composition may be administered with one or more components such as alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene and combinations thereof.

In some variations, the application provides a bioactive composition for enhancing immune function in an individual comprising solubilized Euglena-derived beta-1,3-glucan, wherein the Euglena-derived beta-1,3-glucan is present in an amount from 1 ppm to 10 ppm. In some of these variations, the Euglena-derived beta-1,3-glucan is solubilized by a base.

In some embodiments, the present invention can improve the well-being of an individual. In some variations, the present invention stimulates a macrophage response. Stimulation of the macrophage response is known to activate a cytokine pathway that promotes enhanced general immune system activity. Such a response may be desirable for prevention of infections, treatment of tumors and cancers, or to support a compromised immune system, as would be expected in an immune deficiency syndrome, a patient undergoing surgery or chemotherapy, or a patient with severe burns.

In some variations, the Euglena may be heterotrophically grown. In some variations, the beta-1,3-glucan consists essentially of unbranched beta-1,3-glucan. In some variations, the beta-1,3-glucan does not contain beta-1,6-glycosidic bonds.

In some variations, the composition may be a liquid composition. In other variations, the composition may be a gel composition.

In some variations, the composition may include an additional component such as alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-omithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene and combinations thereof.

In some variations, the composition may include a metal, including for instance an alkali metal, alkaline earth metal, transition metals or nonmetal. In some of these variations, the metal may be iron, magnesium, lithium, zinc, copper, chromium, nickel, cobalt, vanadium, molybdenum, manganese, selenium, and combinations thereof. In some variations, the beta-1,3-glucan and the metal form a complex.

In another aspect, the application provides kits for enhancing the immune function in an individual in need thereof including a bioactive composition provided herein and instructions for use.

### BRIEF DESCRIPTION OF THE DRAWINGS

FIG. 1A shows the structure of a beta-1,3-glucan chain, such as that derived from Euglena. FIG. 1B shows the structure of a beta-1,3-glucan backbone with beta-1,6-glucan side chains, such as that derived from yeast.

FIGS. 2A-2J show cytokine production by murine macrophages treated with 0.15, 0.31, 0.62, 1.25, 2.5, 5, or 10 μg/ml of particulate beta-1,3-glucan derived from Euglena (black bars) or solubilized beta-1,3-glucan derived from Euglena (grey bars) for 48 hours compared to untreated cells and cells treated with 1 μg/ml lipopolysaccharide (LPS).

FIGS. 3A and 3B show the results of flow cytometry analysis of expression of CD86 and MHC II by murine macrophages treated with 0.15, 0.31, 0.62, 1.25, 2.5, 5, or 10 μg/ml of particulate beta-1,3-glucan derived from Euglena (black bars) or solubilized beta-1,3-glucan derived from Euglena (grey bars) for 48 hours compared to untreated cells and cells treated with 1 μg/ml lipopolysaccharide (LPS).

FIGS. 4A-4J show cytokine production by murine dendritic cells treated with 0.15, 0.31, 0.62, 1.25, 2.5, 5, or 10 μg/ml of particulate beta-1,3-glucan derived from Euglena (black bars) or solubilized beta-1,3-glucan derived from Euglena (grey bars) for 48 hours compared to untreated cells and cells treated with 1 μg/ml lipopolysaccharide (LPS).

FIGS. 5A and 5B show the results of flow cytometry analysis of expression of CD86 and MHC II by murine dendritic cells treated with 0.15, 0.31, 0.62, 1.25, 2.5, 5, or 10 μg/ml of particulate beta-1,3-glucan derived from Euglena (black bars) or solubilized beta-1,3-glucan derived from Euglena (grey bars) for 48 hours compared to untreated cells and cells treated with 1 μg/ml lipopolysaccharide (LPS).

FIGS. 6A-6H show cytokine production by immune cells purified from mice treated with 500 μg particulate beta-1,3-glucan (pAG500), 500 μg solubilized beta-1,3-glucan (sAG500), 125 μg solubilized beta-1,3-glucan, or 50 μg solubilized beta-1,3-glucan per day by oral gavage for seven days.

### DETAILED DESCRIPTION

The inventors have surprisingly shown that administration of solubilized beta-1,3-glucan derived from Euglena can be used to promote immune system health and to treat and/or prevent disease in animals, including humans at lower levels than particulate beta-1,3-glucan. For example, solubilized beta-1,3-glucan derived from Euglena can be used to modulate an autoimmune response, blood sugar levels, cholesterol level, an infection, or inflammation.

There are several advantages to using solubilized beta-1,3-glucan derived from Euglena in accordance with the methods provided herein. Solubilized beta-1,3-glucan derived from Euglena is able to modulate immune response in an individual at concentrations that are lower than those required for particulate beta-1,3-glucan. Because a lower concentration of solubilized beta-1,3-glucan is effective for enhancing immune function, a variety of administration options are available, such as by drinking a small amount of solubilized beta-1,3-glucan dissolved in a liquid, such as water. These additional administration options may not be possible with the higher concentrations of particulate beta-1,3-glucan that are required. Moreover, a homogenous dose may be difficult to achieve with a suspension of particulate beta-1,3-glucan. Furthermore, solubilized beta-1,3-glucan is more cost-effective for enhancing immune function in an individual because less beta-1,3-glucan is required to provide an immunomodulatory effect.

### Definitions

The term “Euglena” is understood to mean any species or strain within the Euglena genus, unless otherwise specified. In a preferred embodiment, the Euglena is Euglena gracilis, but other Euglena species are contemplated.

The term “derived from” means that the compound of material originated from a particular source. For example, beta-1,3-glucan derived from Euglena indicates that the beta-1,3-glucan originated from Euglena. The beta-1,3-glucan may be associated with the Euglena or may be purified and hence separated from the Euglena.

The term “modulate” as used herein means to effect or change and may be used interchangeably with “enhance.” For example, “modulating an immune response” means increasing or decreasing an immune response and is synonymous with “enhancing immune function.”

The terms “subject”, “patient”, and “individual” are used synonymously herein to describe any human or animal (including, but not limited to a dog, cat, rodent, horse, sheep, cow, pig, goat, donkey, llama, fish, chicken or rabbit).

The terms “treat,” “treating,” and “treatment” are used synonymously herein to refer to any action providing a benefit to a patient at risk for or afflicted with a disease state or condition, including improvement in the condition through lessening, inhibition, suppression, or elimination of at least one symptom, delay in progression of the disease, or inhibition of the disease. Treatment as used herein also includes prophylactic treatment which can prevent or delay a disease or disorder from occurring. Treatment as used herein may also refer to improving the well-being of a human or animal.

As used herein, the singular forms “a,” “an,” and “the” include plural referents unless the context clearly dictates otherwise. Thus, for example, reference to “a factor” refers to one or mixtures of factors, and reference to “the method of treatment” includes reference to equivalent steps and methods known to those skilled in the art, and so forth.

Before explaining the various embodiments of the disclosure, it is to be understood that the invention is not limited in its application to the details of construction and the arrangement of the components set forth in the following description. Other embodiments can be practiced or carried out in various ways. Also, it is to be understood that the phraseology and terminology employed herein is for the purpose of description and should not be regarded as limiting the inventions described in any way.

Throughout this disclosure, various publications, patents and published patent specifications are referenced. Where permissible, the disclosures of these publications, patents and published patent specifications are hereby incorporated by reference in their entirety into the present disclosure to more fully describe the state of the art.

Compositions Comprising Solubilized Beta-1,3-Glucan

Properties of Solubilized Beta-1,3-Glucan Derived from Euglena

Beta-1,3-glucan derived from Euglena is structurally distinct from beta glucans produced by other organisms and particulate beta-1,3-glucan in terms of its carbohydrate branching structure and three dimensional structure. Moreover, solubilized beta-1,3-glucan derived from Euglena is distinct from particulate beta-1,3-glucan derived from Euglena in terms of its three dimensional structure and bioactivity.

### Carbohydrate Branching Structure

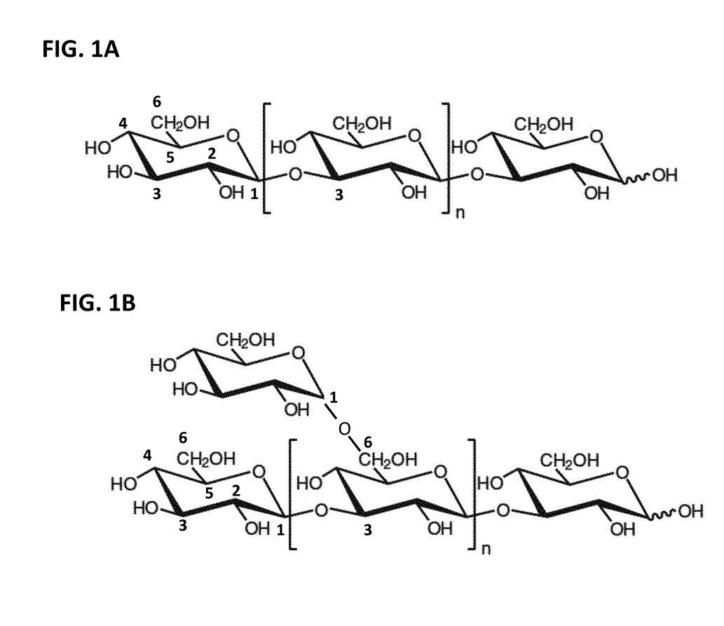

Beta glucans produced by different organisms can vary substantially in the carbohydrate branching structure of the polymer. For example, beta glucan derived from algae such as Euglena has almost exclusively 1,3-glycosidic bonds and no 1,6-glycosidic bonds (FIG. 1A). In contrast, beta glucan derived from yeast has a mixture of beta-1,3- and beta-1,6-glycosidic linkages, generally with a beta-1,3-glucan backbone that includes beta-1,6-side chains (2-3 glucose units long) every 10-30 glucose monomers (FIG. 1B). Beta glucan derived from oats or barley has a mixture of beta-1,3- and beta-1,4-glycosidic linkages. Beta glucan derived from kelp (e.g., Laminaria) has a mixture of beta-1,3- and beta-1,6-glycosidic linkages.

A substantial portion of the beta glucan produced by Euglena is located in the algal cytoplasm as paramylon bodies, and is commonly referred to as “paramylon.” Paramylon derived from Euglena has a linear structure with almost exclusively beta-1,3-glucan with no beta-1,6-side branches. The unbranched nature of paramylon is an important distinction compared to other sources of beta glucans when considering its use in immune support applications. Paramylon produced by Euglena is considered to be one of the more structurally simple of the beta glucans, with few glycosyl side chains. This is in direct contrast to laminaran, lentinan, scleroglucan, schizopylann, or yeast-derived beta glucans that have 1,4- or 1,6-linked side chains.

A study of the branching structure of paramylon reveals its unique structure, and is disclosed in U.S. Patent Publication No. 2013/0216586, which is incorporated in its entirety herein. After isolating paramylon from whole Euglena cells, a linkage analysis was performed to determine the relative amounts of each type of bond between glucose monomers. The results are summarized in Table 1.

TABLE 1
Linkage Analysis of Two Paramlyon Samples
Extracted from Euglena gracilis.

| | Sample 1 | Sample 2 |
|---|---|---|
| GLYCOSYL RESIDUE | (%) | (%) |
| Terminally-linked glucopyranosyl residue (t-glc) | 0.34 | 0.3 |
| 3-linked glucopyranosyl residue (3-glc) | 93.03 | 94.1 |
| 4-linked glucopyranosyl residue (4-glc) | 2.25 | 2.4 |
| 2,3-linked glucopyranosyl residue (2,3 glc) | 3.47 | 2.3 |
| 3,6-linked glucopyranosyl residue (3,5-glc) | 0.36 | 0.8 |
| 2,3,4-linked glucopyranosyl residue (2,3,4-glc) | 0.55 | 0.1 |
| TOTAL | 100.0 | 100.0 |

This linkage analysis indicates that both paramylon samples are mainly composed of 3-linked glucopyranosyl residues. Minor amounts of 4-linked and 2,3-linked glucopyranosyl residues were found along with negligible amounts of 3,6-linked, terminal and 2,3,4-linked glucopyranosyl residues. These data confirm that paramylon is comprised mostly of a linear, unbranched beta-1,3-glucan.

Beta-1,3-glucan is the form of beta glucan that predominantly binds to receptors on the surface of immune system cells, such as Dectin-1 (a macrophage receptor) and complement receptor 3. Beta-1,3-glucan can also be fermented by microflora in an individual's intestine, which may result in the production of beneficial metabolites like short chain fatty acids that may affect the animal's health.

Beta-1,3-glucan derived from Euglena useful for the methods described herein contains about 85% or more beta-1,3-glycosidic linkages, about 87% or more beta-1,3-glycosidic linkages, about 90% or more beta-1,3-glycosidic linkages, about 91% or more beta-1,3-glycosidic linkages, about 92% or more beta-1,3-glycosidic linkages, about 93% or more beta-1,3-glycosidic linkages, or about 94% or more beta-1,3-glycosidic linkages.

### Three-Dimensional Structure

The three-dimensional structure and folding of beta-1,3-glucan can affect the bioavailability, surface area, and overall efficacy in immune stimulation applications. Specifically, the unique three-dimensional structure of solubilized beta-1,3-glucan results in a much more potent form than the three-dimensional structure of particulate beta-1,3-glucan as described below.

In beta-1,3-glucan chains, the structure is governed by the glycosidic linkage pattern. Because the chair-form ring of glucopyranosyl is rather rigid, most of the flexibility of the glucan chain arises from rotations around the bonds of the glycosidic linkages. X-ray crystallography and spectroscopy techniques indicate that particulate beta-1,3-glucan has a triple-helix backbone. The triple-helix structure is stable over a broad range of temperatures at a neutral pH, resulting in a polymer that is water insoluble.

Solubilization of beta-1,3-glucan results in disruption and unwinding of the triple-helix structure of particulate beta-1,3-glucan. Soluble beta-1,3-glucan comprises unwound, free, individual chains of beta-1,3-glucan in solution. Moreover, different immunological effects can be obtained that are related to the beta-1,3-glucan conformation, be it the native state, denatured, or denatured and re-natured. Specifically, the compositions provided herein comprise solubilized beta-1,3-glucans that are in a confirmation that makes them especially effective for enhancing an immune response compared to, for example, beta glucans in a particulate form. The conformation of the beta glucan and its resulting solubility may also affect how it is delivered. For example, water soluble beta-1,3-glucan can be injected intravenously, which may not be possible for particulate beta-1,3-glucan.

As described herein, solubilized beta-1,3-glucan refers to beta-1,3-glucan that has been exposed, at one point in time, to a solubilizing agent, such as base, heat, or detergent, which causes the beta-1,3-glucan to unwind and facilitates solubilization in a solution, such as an aqueous or organic solvent. In this way, the solubilizing agent is effectively a denaturing agent. Although subsequent exposure of the solubilized beta-1,3-glucan to certain conditions, for example neutral pH, may afford a semi-solid, colloidal, or gel-like preparation, solubilized beta-1,3-glucan as referred to herein may also describe semi-solid, colloidal, or gel-like preparations that are derived from fully or partially beta-1,3-glucan that has been solubilized in a solution.

Purity Level of Beta-1,3-Glucan

The level of purity of a beta glucan compound has been determined to have an effect on efficacy, possibly stemming from other material present that inhibits the interaction between the beta glucan and immune cells. Because the beta-1,3-glucan produced by Euglena is stored in water-insoluble granules of about 0.5 to 2.0 microns in size, using the methods described herein, beta-1,3-glucan can be easily isolated in the form of granules from Euglena cells. Specifically, beta-1,3-glucan can be isolated by lysing the Euglena cells, for example by sonication or high pressure homogenization, and then using filtration or gravity separation to isolate the beta-1,3-glucan particles.

As a result, the purity of the beta-1,3-glucan derived from Euglena is very high relative to common preparations of beta glucans from yeast and other organisms. Using the methods described herein, purity levels greater than 95 weight percent can be obtained on an as-received basis. In some embodiments, purity levels greater than 99 weight percent are obtained on an as-received basis. In comparison, the highest-grade yeast-derived beta glucans can rarely achieve greater than 90% purity and most are about 70-80% purity. Moreover, high purity beta-1,3-glucan can be achieved more cost-effectively when produced by Euglena than with yeast-derived glucans due to the ease of separation resulting from the lack of a cell wall in Euglena and easy recovery of the beta-1,3-glucan granules. Finally, since no harsh chemicals (e.g., strong acids and bases or solvents) are required to recover the beta-1,3-glucan derived from Euglena, the beta-1,3-glucan can be recovered in its native form without modifying its chemical composition and configuration. In some embodiments, purified beta-1,3-glucan derived from Euglena is more that 85% pure, more than 90% pure, more than 92% pure, more than 94% pure, more than 95% pure, more than 96% pure, more than 97% pure, more than 98% pure, or more than 99% pure.

Solubilized Beta-1,3-Glucan

In some variations, insoluble beta-1,3-glucan derived from Euglena can be solubilized to increase its bioactivity. Bioactivity as used herein may include any change in an individual's physiology, health, or well-being, such as those provided herein. For example, bioactivity may include disease treatment or prevention. A bioactive composition may also result in enhanced immune function, or modulation of an immune response, blood sugar level, cholesterol level, an infection, or inflammation. Bioactivity can be measured through monitoring certain biomarkers, such as relevant protein, RNA, or cytokine level, including those provided herein. Bioactivity can also be measured as an increased response rate of a patient population to a particular treatment, decreased mortality, increased longevity, or a change in other clinical indicators and/or symptoms of a disease or dysfunction. For example, a bioactive compound or composition may result in decreased joint pain, stiffness, swelling, lower cholesterol, decreased antibody titers, increased antibody titers, decreased blood sugar level, or increased blood sugar level.

Various agents may be used to facilitate the solubilization of beta-1,3-glucan in a solvent, such as an aqueous or organic solvent. For instance bases, chaotropic agents, and detergents may be used to facilitate solubilization. In at least one embodiment, a base is used to facilitate the solubilization of beta-1,3-glucan derived from Euglena in a solvent. Bases are molecules that are able to accept protons. Typically, bases are agents that increase the pH of an aqueous solution. Basic solutions that can be used to solubilize the beta-1,3-glucan have a pH of greater than 7.0 and are also known as alkaline or caustic solutions. Suitable bases include but are not limited to alkali and alkaline earth metal bases, as well as alkali salts of weak or strong acids. For instance, suitable bases include but are not limited to sodium hydroxide, potassium hydroxide, calcium hydroxide, magnesium hydroxide, sodium carbonate, and ammonium. In some variations, a strong base (completely dissociated in solution; sodium hydroxide, potassium hydroxide, calcium hydroxide, for instance) is used to facilitate the solubilization of the beta-1,3-glucan. In other variations, a weak base (partially dissolved in solution; magnesium hydroxide, sodium carbonate, and ammonium, for instance) is used to facilitate the solubilization of the beta-1,3-glucan.

Various amounts of a base can be used to aid the solubilization of the beta-1,3-glucan in a solution. In some variations, an amount of base effective to solubilize the beta-1,3-glucan is used. For example, in at least one embodiment 0.2M to 10M of a base can be used. In some variations, beta-1,3-glucan is solubilized in a solution with 2M, 1.9M, 1.8M, 1.7M, 1.6M, 1.5M, 1.4M, 1.3M, 1.2M, 1.1M, 1M, 0.9M, 0.8M, 0.7M, 0.6M, 0.5M, 0.4M, 0.3M, or 0.2M sodium hydroxide. In variations when a base is used to solubilize the beta-1,3-glucan, an acid (molecule or ion capable of releasing a proton) can be added to neutralize the pH of the solution after the beta-1,3-glucan is solubilized with a base. By way of a non-limiting example, following solubilization with the aid of a base, the pH of a solution comprising beta-1,3-glucan can be adjusted using HCl.

In some variations, beta-1,3-glucan is solubilized using a chemical other than a base. For example, in some of these variations, beta-1,3-glucan is solubilized by incubating with a chaotropic agent. Chaotropic agents are molecules that disrupt the hydrogen bonding network between water molecules. Chaotropic agents destabilize macromolecules by removing surrounding water molecules. Exemplary chaotropic agents include but are not limited to urea, guanidine, butanol, ethanol, guanidium chloride, lithium perchlorate, sodium perchlorate, lithium acetate, magnesium chloride, phenol, propanol, sodium dodecyl sulfate, and thiourea. In at least one embodiment, the beta-1,3-glucan is solubilized by incubation with 8M urea or 6M guanidine hydrochloride.

In other variations, beta-1,3-glucan is solubilized in a solution by incubating with a solvent such as dimethyl sulfoxide, dimethylformamide, methanol, acetone, or acetonitrile. In yet other variations, a solubilizing agent such as a detergent, for instance a zwitterionic, ionic, or non-ionic detergent is used to facilitate solubilization. Exemplary detergents include but are not limited to Tween, octyl-glucose, CHAPS, and CHAPSO.

Application of heat is another method to increase the solubility of beta-1,3-glucan. For example, an amount of beta-1,3-glucan ranging from 0.1% to 10% (as measured by mass) can be combined with boiling water or other aqueous solution for at least 10 minutes and cooled to room temperature. The result is a solubilized beta glucan solution having a viscosity related to the amount of beta glucan. The viscosity can be tailored based upon the resulting chain length and/or the concentration of the beta glucan. For example, it is possible to heat beta glucan in an aqueous solution to produce a viscosity of about 600 g/cm2 or more for certain applications. Such solutions can have a gel-like consistency.

In some variations, it may be beneficial to alter the beta glucan chain length using enzymes, catalysis, heat, sonication, or combinations thereof. In some variations, altering the chain length of beta-1,3-glucan may improve solubility of the beta-1,3-glucan. Additionally, it can be beneficial to start with a highly pure linear source of beta-1,3-glucan, such as beta-1,3-glucan derived from Euglena gracilis, in order to achieve a desired range of optimal target chain lengths.

One non-limiting example of a process for achieving a beta-1,3-glucan with a shorter chain length includes the following steps.
1) Start with a beta-1,3-glucan derived from Euglena having an average molecular weight of about 500 kDa. This corresponds to a linear chain of approximately 3,000-4,000 glucose subunits.
2) Optionally, a pre-preparation of the beta-1,3-glucan may be required to unwind or unzip the crystalline beta-1,3-glucan structure that occurs in paramylon derived from Euglena.
3) Cleave the molecule, where one example of a target molecular weight includes approximately 5 to 20 kDa, or approximately 30 to 250 glycosidic subunits. In some cases, it may be beneficial to cleave the molecule prior to unwinding or unzipping the 3D structure of the beta glucan chain such as to expose only a portion of the bonds between glycosidic subunits. Cleavage techniques can include:
   a. Enzymatic cleavage, such as by using beta glucanase or a similar enzyme.
   b. Ultrasonification, either on a plate or by combining with ultrasonified micro-particles or nano-particles.
   c. Use of a catalyst.
   d. Heat.
   e. Use of energy-transferring wavelengths emitted from a device such that the waves are absorbed by the bonds linking the subunits, where sufficient energy is applied to break a portion of the bonds.
4) An optional separation or purification step can be performed where a relatively homogeneous product is desired and the resulting chain lengths of the cleaved beta glucan are not uniform. Size selection of beta-1,3-glucan can include:
   a. Centrifugation or sedimentation, where heavier molecules are more dense and have less relative surface area.
   b. Filtration, e.g. using Millipore-type or other filters or a series of such filters, to separate or isolate the target beta-1,3-glucan chain-length.
   c. Chromatography, such as size-exclusion chromatography.
   d. Electrophoresis, including gel electrophoresis.

Solubilization can be performed at a range of temperatures. For example, solubilization may be performed at temperatures between 4° C. and 200° C. In some variations, solubilization is performed by incubation at room temperature. In other variations, solubilization is facilitated by incubation at 30° C., 40° C., 50° C., 60° C., 70° C., 80° C., 90° C., 100° C., 110° C., or 120° C.

Solubilization can be performed at a range of pressures. For example, solubilization may be performed at pressures between 0.5 atm and 100 atm. In some variations, solubilization is performed by incubation at ambient pressure (1 atm). In other variations, solubilization is performed by incubation at 1.5 atm, 2 atm, 3 atm, 4 atm, 5 atm, 10 atm, 25 atm, 50 atm, 75 atm, or 100 atm. In some variations, solubilization is performed by incubation in an autoclave.

In some variations, beta-1,3-glucan is solubilized by incubating with a solubilizing agent such as a base, chaotropic agent, solvent, or detergent for varying amounts of time. For example, in some variations, the beta-1,3-glucan is solubilized by incubating with a solubilizing agent for between 1 minute and 5 hours. In some of these variations, the beta-1,3-glucan is solubilized by incubating with a solubilizing agent for about 30 minutes, about 60 minutes, about 90 minutes, about 120 minutes, about 150 minutes, about 180 minutes, about 210 minutes or about 240 minutes or 5 hours or more.

Various combinations of the parameters of temperature, the presence of a solubilizing agent, and time can be used to solubilize the beta-1,3-glucan in a solution, such as an aqueous or organic solvent, some of which are exemplified in Table 2. For example, the beta-1,3-glucan can be solubilized by incubating with 1M NaOH solution at room temperature for two hours. In some variations, the beta-1,3-glucan is solubilized by incubating with 1M NaOH at greater than room temperature for less than two hours. In some variations, the beta-1,3-glucan is solubilized by incubating with 1M NaOH at 60° C. for 30 minutes. In some variations, the beta-1,3-glucan is solubilized by incubating with 0.5M NaOH at 90° C. for 10 minutes. In some variations, the beta-1,3-glucan is solubilized by incubating with 0.5M NaOH at room temperature for 4 hours.

TABLE 2
Exemplary solubilization conditions.

| | | Concen- | | |
|---|---|---|---|---|
| | | trations of | | |
| Solubilizing | | solubilizing | | |
| agent class | Solubilizing agents | agent | Time | Temperature |
| Base | NaOH, KOH, | 0.2-2M | 1-5 | Room |
| | Ca(OH)₂, Ba(OH)₂, | | hours | temperature |
| | Mg(OH)₂, NH₃, | | | or 30-95° C. |
| | NaH₂CO₃ | | | |
| Chaotropic | Urea, guanidine, | 1-10M | 1-5 | Room |
| agents | butanol, ethanol, | | hours | temperature |
| | guanidium chloride, | | | or 30-95° C. |
| | lithium perchlorate, | | | |
| | sodium perchlorate, | | | |
| | lithium acetate, | | | |
| | magnesium chloride, | | | |
| | phenol, propanol, | | | |
| | sodium dodecyl | | | |
| | sulfate, thiourea | | | |
| Solvent | Dimethyl sulfoxide, | 10-100% | 1-5 | Room |
| | dimethylformamide, | | hours | temperature |
| | methanol, acetone, | | | or 30-95° C. |
| | acetonitrile | | | |
| Heat | N/A | N/A | 10 min- | 50-120° C. |
| | | | 5 | |
| | | | hours | |

In some variations, the beta-1,3-glucan is solubilized by incubating with 8M urea at room temperature for two hours. In other variations, the beta-1,3-glucan is solubilized by incubating with 8M urea at greater than room temperature for less than two hours. In some variations, the beta-1,3-glucan is solubilized by incubating with 8M urea at room temperature for two hours. In some variations, the beta-1,3-glucan is solubilized by incubating with 8M urea at greater than room temperature for less than two hours. In some variations, the beta-1,3-glucan is solubilized by incubating with 6M guanidine at room temperature for two hours. In some variations, the beta-1,3-glucan is solubilized by incubating with 6M guanidine at greater than room temperature for two less than two hours. In some variations, the beta-1,3-glucan is solubilized by incubating with 50% DMSO at room temperature for two hours. In some variations, the beta-1,3-glucan is solubilized by incubating with 50% DMSO at greater than room temperature for less than two hours.

Solubilized beta-1,3-glucan may be more bioactive than the particulate form of beta-1,3-glucan, such that a lower amount of solubilized beta-1,3-glucan is required to produce a physiological benefit to an individual. For example, compositions comprising solubilized beta-1,3-glucan may comprise less than ½, less than ¼, less than 1/10, or less than 1/100 of the amount of beta-1,3-glucan provided in compositions comprising particulate beta-1,3-glucan. In some variations, the present invention includes compositions comprising solubilized beta-1,3-glucan at lower concentrations than particulate beta-1,3-glucan. For example, compositions comprising solubilized beta-1,3-glucan may contain from about 200 to about 1 ppm of beta-1,3-glucan. In some of these variations, compositions comprising solubilized beta-1,3-glucan contain from 1 ppm to 100 ppm, from 1 ppm to 50 ppm, from 1 ppm to 25 ppm, from 1 ppm to 10 ppm, or from 1 ppm to 5 ppm of beta-1,3-glucan. In some variations, the composition comprises an amount of solubilized beta-1,3-glucan effective to enhance immune function when administered to an individual.

In some variations, the composition comprising beta-1,3-glucan derived from Euglena has a pH that is suitable for administration to an individual. The appropriate pH will depend upon the particular formulation employed and desired mode of administration. For example, solutions for intravenous administration may have a pH between 4.5 and 8, or preferably between pH of 7 and 8. In some variations, the composition has a pH of greater than 7. In other variations, the composition has a pH of less than 7. In yet other variations, the composition has a pH of approximately 7. Appropriate buffers may be used to maintain the desired pH. For example, buffering solutions comprising phosphate, citrate, bicarbonate or acetate may be employed.

Solubilized beta-1,3-glucan can be stored for extended periods under alkaline conditions (i.e. pH of more than 7) to inhibit microbial growth. It may be beneficial, therefore, to solubilize the beta-1,3-glucan with a base and later neutralize the solution just prior to use to prevent microbial contamination during storage. A salt, such as NaCl, may form after neutralization of a solution of solubilized beta-1,3-glucan. In some variations, salts are removed to achieve an appropriate osmolarity. Salts can be removed, for example, by dialyzing a solution comprising solubilized beta-1,3-glucan with a solution having the desired osmolarity.

In some variations, the composition comprising beta-1,3-glucan may further comprise additional components for enhancing an immune response. For example, alpha tocopherol, cholecalciferol, zinc, chromium, selenium, arginine, ascorbic acid, alklyglcerol, caffeine, kava kava, curcuma longa, Spirulina, Chlorella, stevia, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, olive leaf, Sambucus, Umcka, panthothenic acid, quercetin, alpha lipoic acid, essential oils, fish oils, spices and their derivatives, and pterostilbene can be included in the composition comprising beta-1,3-glucan.

In some variations, the composition comprising beta-1, 3-glucan may be administered with one or more additional components for enhancing an immune response. For example, alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-omithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene and combinations thereof may be administered with the composition comprising beta-1,3-glucan. For example the beta-1,3-glucan composition may be administered simultaneously or sequentially with any of the additional agents included herein.

Compositions comprising Euglena-derived beta-1,3-glucan may further comprise a metal. In some of these variations, the metal may include iron, magnesium, lithium, zinc, copper, chromium, nickel, cobalt, canadium, molybdenum, manganese, and/or selenium. In some variations, the metal is associated with the beta-1,3-glucan. In some variations, the metal and the beta-1,3-glucan form a complex.

Compositions comprising solubilized Euglena-derived beta-1,3-glucan may further comprise a pharmaceutically acceptable excipient. Example pharmaceutically acceptable excipients include fillers, binders, coatings, preservatives, lubricants, flavoring agents, sweetening agents, coloring agents, surfactants, solvents, buffering agents, chelating agents, or stabilizers. Examples of pharmaceutically acceptable fillers include cellulose, dibasic calcium phosphate, calcium carbonate, microcrystalline cellulose, sucrose, lactose, glucose, mannitol, sorbitol, maltitol, pregelatinized starch, corn starch, and potato starch. Examples of pharmaceutically acceptable binders include polyvinylpyrrolidone, starch, lactose, xylitol, sorbitol, maltitol, gelatin, sucrose, polyethylene glycol, methyl cellulose, and cellulose. Examples of pharmaceutically acceptable coatings include hydroxypropyl methylcellulose (HPMC), shellac, corn protein zein, and gelatin. Examples of pharmaceutically acceptable disintegrants include polyvinylpyrrolidone, carboxymethyl cellulose, and sodium starch glycolate. Examples of pharmaceutically acceptable lubricants include polyethylene glycol, magnesium stearate, and stearic acid. Examples of pharmaceutically acceptable preservatives include methyl parabens, ethyl parabens, propyl paraben, benzoic acid, and sorbic acid. Examples of pharmaceutically acceptable sweetening agents include sucrose, saccharine, aspartame, or sorbitol. Examples of pharmaceutically acceptable buffering agents include carbonates, citrates, gluconates, acetates, phosphates, or tartrates.

In some variations, compositions comprising solubilized beta-1,3-glucan can be formulated as a liquid or a gel. For example, the solubilized beta-1,3-glucan may be dissolved in a beverage, such as in water or provided in a nutritional shake. In some variations, compositions comprising beta-1,3-glucan may be formulated as a liquid that is suitable for IV administration or subcutaneous administration. The composition including the beta-1,3-glucan can be formulated as a concentrate, which is sufficiently storage stable for commercial use and which is diluted, for example with water, before use. Alternatively, each component of the composition can be formulated as a separate concentrate for mixing and dilution prior to use. Liquid compositions may also be ready for immediate use. In other variations, the solubilized beta-1,3-glucan is provided in a gel capsule or an edible gel. In yet further variations, the solubilized beta-1,3-glucan is formulated for topical administration, for example as a topical gel or cream.

Methods of Enhancing Immune Function

The compositions provided herein comprising solubilized beta-1,3-glucan derived from Euglena may be administered to an individual to enhance immune function. Administration of the solubilized beta-1,3-glucan derived from Euglena to the subject may result in a measureable increase of cytokine production, increased expression of cell surface activation markers on immune cells, increased antibody titers, and increased activity of immune system cells (e.g., rates of phagocytosis and natural killer cell cytotoxicity), demonstrating enhanced immune function. Subjects administered solubilized beta-1,3-glucan derived from Euglena may also demonstrate an enhanced response to infection.

Various aspects of immune function can be improved or enhanced by administering a composition comprising solubilized beta-1,3-glucan derived from Euglena. For example, the composition may stimulate or increase an immune response. The composition comprising solubilized beta-1,3-glucan may increase an immune response to prevent or reduce an infection. In some variations, the infection is a bacterial, fungal, or viral infection. Administration of compositions comprising solubilized beta-1,3-glucan may result in increased activity of both innate and adaptive immune functions. For example, administration of the compositions may result in an increase in phagocytosing neutrophils, natural killer cell cytotoxicity, and antibody production. The compositions comprising solubilized beta-1,3-glucan can be administered to a subject that has an infection to treat the infection or prophylactically administered to a subject to limit the risk of infection. These advances in the treatment or prophylactic treatment are particularly important for bacterial infections due to the risk of antibiotic resistant bacteria, including methicillin-resistant Staphylococcus aureus (MRSA).

In other variations, enhancing immune function comprises decreasing the immune response, for example, to modulate an autoimmune response or to treat inflammation. For example, the compositions containing solubilized beta-1,3-glucan derived from Euglena can be administered to a subject, including a human, to modulate an autoimmune response associated with diabetes, Crohn's disease, rheumatoid arthritis, fibromyalgia, systemic lupus erythematosus, glomerulonephritis, scleroderma, or multiple sclerosis. In some embodiments, the composition is prophylactically administered to limit the progression of diabetes, Crohn's disease, rheumatoid arthritis, fibromyalgia, systemic lupus erythematosus, glomerulonephritis, scleroderma, or multiple sclerosis. In some variations, the composition decreases inflammation associated with intestinal inflammation. For example, the composition may be useful for treating conditions such as inflammatory bowel disease, colitis, and Crohn's disease. In some variations, the composition decreases inflammation associated with allergies. In other variations, the method decreases an autoimmune response associated with diabetes.

The compositions containing solubilized beta-1,3-glucans derived from Euglena can also be administered to a subject to modulate blood sugar levels in the subject. After administration of the compositions containing solubilized beta-1,3-glucans, postprandial blood sugars are generally lower than without the administration of the beta-1,3-glucan. The modulation of blood sugars, particularly postprandial blood sugars, is important for general diabetes care and management in both Type I and Type II diabetics. Blood sugar levels can be measured using the A1C test, which reflects average blood sugar levels of the past two to three months. Specifically, the A1C test measures the percentage of hemoglobin that is coated with sugar (i.e. glycated). The compositions and pharmaceutical compositions containing solubilized beta-1,3-glucan derived from Euglena as described herein are therefore useful to treat hyperglycemia in a diabetic. In some embodiments, compositions containing solubilized beta-1,3-glucan derived from Euglena as described herein are prophylactically administered to a subject to limit hyperglycemia.

Administration of compositions comprising solubilized beta-1,3-glucans derived from Euglena as described herein can be administered to a subject, including a human, to modulate inflammation in the subject. The administered beta-1,3-glucan functions to suppress the production of inflammatory cytokines, resulting in a modulated inflammatory response in the subject. In some embodiments, the inflammation is associated with allergies, asthma, or intestinal inflammation. The compositions or pharmaceutical formulations comprising beta-1,3-glucans derived from Euglena as described herein can be administered to a subject to treat inflammation, such as allergies, asthma, or intestinal inflammation. Additionally, the compositions or pharmaceutical formulations comprising beta-1,3-glucans derived from Euglena as described herein can be prophylactically administered to a subject to limit inflammation, such as allergies, asthma, or intestinal inflammation.

In yet other variations, enhancing immune function may comprise modulating cholesterol level. In some of these variations, the composition comprising solubilized beta-1,3-glucans derived from Euglena are useful for treating hyperlipidemia. Hyperlipidemia, or abnormally high blood cholesterol or triglyceride levels, creates substantial risk for heart attacks and cardiovascular disease. Hyperlipidemia may result from genetic factors or certain health or lifestyle factors, including a high-fat or high-cholesterol diet, obesity, or lack of regular exercise. Hyperlipidemia includes any condition resulting in elevated blood cholesterol (i.e., hypercholesterolemia) or blood triglyceride (hypertriglyceridemia) levels. Cholesterol and triglycerides are associated with lipoproteins, including low-density lipoprotein (LDL) and high-density lipoprotein (HDL). LDL, which is frequently referred to as “bad” cholesterol, collects in the walls of blood vessels and can lead to plaque growth and atherosclerosis. In contrast, HDL (often referred to as “good” cholesterol) transfers fats away from cells, artery walls, and tissues through the bloodstream. Increasing concentrations of HDL particles are associated with decreasing accumulation of atherosclerosis within the walls of arteries. Solubilized beta-1,3-glucan derived from Euglena can be administered to a subject, including a human, to treat hyperlipidemia or prophylactically administered to a subject at risk for hyperlipidemia. Solubilized beta-1,3-glucan derived from Euglena can be administered to a subject, including a human, to lower LDL. Solubilized eta-1,3-glucan derived from Euglena can be administered to a subject, including a human, to increase HDL. A person at risk for hyperlipidemia can include, but is not limited to, a person who has been previously diagnosed with hyperlipidemia, a person with a high-fat or high-cholesterol diet, or a person with one or more parents with hyperlipidemia.

Solubilized beta-1,3-glucan derived from Euglena can also be administered to a subject, including a human, to treat non-alcoholic fatty liver disease (NAFLD), or prophylactically administered to a subject at risk for NAFLD. Closely associated with obesity and type 2 diabetes, NAFLD is known to be a major risk factor for cardiovascular diseases.

Solubilized beta-1,3-glucan derived from Euglena can be administered to a subject, including a human, to treat metabolic syndrome, or prophylactically administered to a subject at risk for metabolic syndrome. Metabolic syndrome refers to a cluster of conditions including increased blood pressure, high blood sugar levels, excess body fat, abnormal cholesterol levels that occur together to increase risk of heart disease, stroke, and diabetes.

In some variations, the well-being of an animal can be improved by administering compositions comprising solubilized beta-1,3-glucan derived from Euglena. Well-being includes enhancement of one or more of the following aspects: weight gain, conversion efficiency of food to live weight, behavior, disease resistance, stress tolerance, reduced mortality rates, and improved immune function as described in US2013/0216586, which is herein incorporated by reference. In some variations, compositions comprising solubilized beta-1,3-glucan derived from Euglena decrease infectious diseases such as avian pox, botulism, cholera, bronchitis, infectious coryza, Mareks disease, moniliasis, mycoplasmosis, Newcastle disease, omphalitis, pullorum, foot and mouth disease, brucellosis, equine encephalitis, swollen head syndrome, staph infection, nematode infection, trematode infection, fungal infection, and tuberculosis. In other variations, compositions comprising solubilized beta-1,3-glucan prevent an immune-related disease such as mastitis, systemic lupus erythematosus (SLE), autoimmune hemolytic anemia and thrombocytopenia, autoimmune myasthenia gravis, and diabetes mellitus, or toxic epidermal necrolysis.

Methods to evaluate use of the present compositions in animal feed include measuring increases in antibody titers, measuring increases in the activity of immune system cells (e.g., rates of phagocytosis and natural killer cell cytotoxicity), measuring improvements in feed conversion efficiency, measuring decreased stress, measuring improved weight loss or weight gain, measuring improvements in feed consumption, measuring improvements in average daily gain, performing challenge studies where at least one of the treatment groups is administered a composition as described herein, measuring reduced mortality rates in an animal population, measuring alternations in levels of interleukins or other cytokines which are known to be related to immunological performance, measuring effects on tumor necrosis factor alpha, fluorescently tagging components of the compositions described herein and observing their presence or metabolism in various cell, blood, or tissue samples, performing general histological analysis on animals that are fed a composition described herein, weighing the organs or animals which are fed a composition described herein, or any other analysis that demonstrates a significant effect on animals when they are fed one or more of the compositions described herein.

Cytokines are small proteins released by immune cells that play a key role in cell signaling in response to infection, immune response, and inflammation. Some cytokines promote an inflammatory response, and are known as pro-inflammatory cytokines. Examples of pro-inflammatory cytokines include IL-1, IL-6, IL-8, IL-11, and TNF-11. Other examples of pro-inflammatory mediators include Granulocyte-macrophage colony-stimulating factor (GM-CSF), Interferon gamma (IFN-γ), Tumor growth factor beta (TGF-β), leukemia inhibitory factors (LIF), oncostatin M (OSM), and a variety of chemokines that attract inflammatory cells.

For example, IL-1 is an important pro-inflammatory cytokine. IL-1 is a soluble protein having a mass of approximately 17 kilo-Daltons (kD). IL-1 is produced by a variety of cells, for example macrophages, white blood cells, lymphocytes, monocytes, dendritic cells, and accessory cells that are involved in activation of T-lymphocytes and B-lymphocytes. IL-1 is produced during immune responses. A common function of IL-1 (e.g. IL-1 a and IL-1 P) is an increasing of expression of adhesion factors on endothelial cells to enable transmigration of leukocytes (which are immune cells that fight pathogens) to sites of infection. In addition, IL-1 stimulates the hypothalamus thermoregulatory center to cause an increase in body temperature (i.e. a fever). The increased body temperature helps the body's immune system to fight pathogens or infection within the body. In addition, IL-1 is an important mediator of inflammatory response, and is also involved in a range of cellular activities, for example cell proliferation, cell differentiation, and cell apoptosis.

TNF-α is also an important pro-inflammatory cytokine. TNF-α is involved in systemic inflammation and works in tandem with a variety of other cytokines to stimulate the acute phase immune reaction. TNF-α is capable of inducing apoptotic cell death, inducing inflammation, as well as inhibiting tumorigenesis and viral replication. TNF-α and IL-1 commonly work simultaneously and synergistically in stimulating and sustaining inflammation within the body.

Other cytokines inhibit inflammation and are known as anti-inflammatory cytokines. Anti-inflammatory cytokines generally facilitate control or mitigation of the magnitude of inflammation in vivo. Functions of anti-inflammatory cytokines include inhibiting production of proinflammatory cytokines and inhibiting cell activation. Examples of anti-inflammatory cytokines include IL-2, IL-4, IL-10, and IL-13.

Many cytokines that play a role in the immune response are known. For example, exemplary cytokines that may be produced by immune cells as part of an immune response include TNFα, IL-1 IL-2, IL-4, IL-6, IL-7, IL-12, IL-10, IL-11, IL-13, IL-18 IFN-γ IL-1β, IL-23, MCP-1, MIP-1α, TGF-β and RANTES.

Cytokines play a key role in many inflammatory diseases. For example, IL-1□ and IL-1□ are important inflammatory cytokines in rheumatoid arthritis, IL-12 has been shown to be elevated in patients with Crohn's disease, and IL-6 may play a role in ulcerative colitis and Crohn's disease, and multiple sclerosis, among other disorders. Cytokines and Chemokines in Autoimmune Disease: An Overview, Pere Santamaria, Madame Curie Bioscience Database, Landes Bioscience (2013).

Many types of immune cells produce and/or respond to cytokines during immune stimulation. For example, upon stimulation, immune cells such as macrophages, B lymphocytes, T lymphocytes, mast cells and dendritic cells may secrete a number of cytokines to regulate the immune response. Moreover, many of these cells play a role in both increasing and decreasing inflammation. For example, macrophages are a type of white blood cells that plays a role both in stimulating inflammation and in decreasing immune reactions.

One way to measure an enhanced immune response in a subject is by detecting cytokine levels. In some variations, compositions comprising solubilized beta-1,3-glucan increase certain cytokine levels in a subject. In some of these variations, solubilized beta-1,3-glucan is more effective for increasing certain cytokine levels than particulate beta-1,3-glucan. In some variations, compositions comprising solubilized beta-1,3-glucan decrease certain cytokine levels in a subject.

Another way to detect an immune response in a subject is by detecting cell surface receptors whose expression is increased upon immune stimulation. For example, MHC class II molecules are found on antigen-presenting cells such as dendritic cells, mononuclear phagocytes, and B cells. Although MHC II is constitutively expressed on certain antigen presenting cells, its expression can be induced on macrophages upon immune stimulation. For example, LPS stimulation increases MHC II expression in B cells and dendritic cells. (Casals et al. J. Immunology 178(10):6307-6315 (2007). CD86 is a costimulatory molecule that provides signals for T cell activation and stimulation. CD86 is primarily expressed on dendritic cells, macrophages and B-cells. Expression of CD86 may be increased upon immune stimulation.

Various methods can be used to detect expression of cell surface receptors such as MHC II and CD86, which may be induced upon administration of a composition comprising solubilized beta-1,3-glucan to an individual. For example, protein levels may be detected using flow cytometry, immunohistochemistry, western blot, ELISA, or immune-electron microscopy. In other variations, RNA levels may be detected, for example, by northern blot, qPCR, microarray, or fluorescence in situ hybridization.

In some variations, compositions containing solubilized beta-1,3-glucan derived from Euglena may be used to enhance immune function or well-being in a plant as described in US2014/0287917, which is herein incorporated by reference. Plants lack an adaptive immune system like most vertebrates, but have an active innate immune system that is based on the recognition of pathogen-associated molecular patterns (PAMPs). These are conserved molecules that are unique to certain classes of microorganisms. The solubilized beta-1,3-glucan compositions provided here may act as a PAMP that stimulates that immune system of the plant to improve growth rate, desired agricultural product (i.e. the crop), disease resistance, stress tolerance, reduced mortality rates, and improved immune function or quality of the harvested plant material, wherein the “quality of the of the harvested plant material” includes reduction in damage due to harvest, transport and storage, improvement in appearance, and longer shelf life.

Compositions containing solubilized beta-1,3-glucan derived from Euglena are administered in an effective dose to enhance immune function. Such dosing regimens are generally understood as an amount of beta-1,3-glucan per kg body weight for each of the composition or pharmaceutical formulation. In some embodiments, the composition or pharmaceutical formulation is administered to the subject at an effective amount of about 0.1 mg beta-1,3-glucan per kg body weight or more, about 0.25 mg beta-1,3-glucan per kg body weight or more, about 0.5 mg beta-1,3-glucan per kg body weight or more, about 1 mg beta-1,3-glucan per kg body weight or more, about 2 mg beta-1,3-glucan per kg body weight or more, about 5 mg beta-1,3-glucan per kg body weight or more, about 10 mg beta-1,3-glucan per kg body weight or more, about 15 mg beta-1,3-glucan per kg body weight or more, about 25 mg beta-1,3-glucan per kg body weight or more, about 50 mg beta-1,3-glucan per kg body weight or more, about 75 mg beta-1,3-glucan per kg body weight or more, or about 100 mg beta-1,3-glucan per kg body weight or more. In other embodiments, the effective amount of the composition or pharmaceutical composition used to modulate the immune function of the subject, to treat a disease, or for prophylactic administration is between about 0.1 mg beta-1,3-glucan per kg body weight and about 100 mg beta-1,3-glucan per kg body weight, between about 0.1 mg beta-1,3-glucan per kg body weight and about 75 mg beta-1,3-glucan per kg body weight, between about 0.1 mg beta-1,3-glucan per kg body weight and about 50 mg beta-1,3-glucan per kg body weight, between about 0.1 mg beta-1,3-glucan per kg body weight and about 25 mg beta-1,3-glucan per kg body weight, between about 0.2 mg beta-1,3-glucan per kg body weight and about 15 mg beta-1,3-glucan per kg body weight, between about 0.5 mg beta-1,3-glucan per kg body weight and about 10 mg beta-1,3-glucan per kg body weight, between about 1 mg beta-1,3-glucan per kg body weight and about 10 mg beta-1,3-glucan per kg body weight, between about 25 mg beta-1,3-glucan per kg body weight and about 75 mg beta-1,3-glucan per kg body weight, between about 25 mg beta-1,3-glucan per kg body weight and about 50 mg beta-1,3-glucan per kg body weight, between about 50 mg beta-1,3-glucan per kg body weight and about 75 mg beta-1,3-glucan per kg body weight, or between about 75 mg beta-1,3-glucan per kg body weight and about 100 mg beta-1,3-glucan per kg body weight. In some embodiments, the effective amount of the composition or pharmaceutical composition used to modulate the immune function of the subject, to treat a disease, or for prophylactic administration is about 0.1 mg beta-1,3-glucan per kg body weight, about 1 mg beta-1,3-glucan per kg body weight, about 10 mg beta-1,3-glucan per kg body weight, about 25 mg beta-1,3-glucan per kg body weight, about 50 mg beta-1,3-glucan per kg body weight, about 75 mg beta-1,3-glucan per kg body weight, or about 100 mg beta-1,3-glucan per kg body weight.

An effective amount of the composition containing the solubilized beta-1,3-glucan derived from Euglena can be administered to the subject to modulate immune function in a single dose once per day. In some embodiments, an effective amount of a composition comprising beta-1,3-glucan derived from Euglena is administered to a subject as multiple doses per day, for example twice per day or more frequently, three times per day or more frequently, or four times per day or more frequently. In some embodiments, an effective amount of an edible or pharmaceutical composition comprising soluble beta-1,3-glucan derived from Euglena is administered to a subject once per week or more frequently, twice per week or more frequently, three times per week or more frequently, four times per week or more frequently, five times per week or more frequently, or six times per week or more frequently.

Administration of solubilized beta-1,3-glucan derived from Euglena can be oral, such as by administering an edible composition or an oral pharmaceutical formulation, or intravenous, such as by administering an intravenous pharmaceutical formulation. Alternate routes of administration, such as by inhalation, are also contemplated. Typically, the pharmaceutical formulation suitable for inhalation includes purified, solubilized beta-1,3-glucans derived from Euglena, which may be administered by, for example, a nasal spray. The edible composition or pharmaceutical formulation can be administered in combination with one or more statins, nicotinic acid, bile acid resins, fibric acid derivatives, or cholesterol absorption inhibitors to enhance the treatment of hyperlipidemia, for example. The edible composition or pharmaceutical formulation can be administered in combination with anti-inflammatory drugs, immunosuppression drugs, or antibiotics to enhance the treatment of intestinal inflammation, for example.

### Kits

In some variations, the invention provided herein includes kits comprising a composition comprising solubilized beta-1,3-glucan derived from Euglena and instructions for use. For example the kit may comprise a composition comprising solubilized beta-1,3-glucan derived from Euglena an instructions for administering the composition to an individual. In some variations, the kit may comprise one or more containers filled with one or more ingredients of the solubilized beta-1,3-glucan compositions provided herein.

The kit may contain a composition comprising solubilized beta-1,3-glucan and an additional agent. In some of these variations, the additional agent may include alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene and combinations thereof.

Method for Producing Compositions Comprising Beta-1,3-Glucan Derived from Euglena

Euglena is a genus of green algae that naturally grows and reproduces in a photosynthetic state, thus relying on sunlight to for survival. However, large-scale culture of Euglena grown using photosynthesis is difficult and not cost-effective. Moreover, Euglena grown using photosynthesis results in much lower amounts of beta glucan (i.e. less than 20% of the total Euglena cell mass). Accordingly, the Euglena useful for the methods and compositions described herein can be grown by fermentation in large fermentation tanks. Generally, the fermenting Euglena cultures are heterotrophically grown, with little or no ambient light, relying on provided nutrients to synthesize the beta-1,3-glucan and other cellular components. Euglena grown using fermentation can grow to a greater cell density than naturally occurring or photosynthetic Euglena cultures, thereby producing higher amounts of beta-1,3-glucan.

Preferably, the Euglena useful for the methods disclosed herein is grown in a controlled environment such that the Euglena will remain the dominant microorganism in the environment. Controlled growth of any organism is difficult, as many contaminating organisms are capable of competing for the same biological resources (e.g., nutrients, micronutrients, minerals, and/or organic energy). Many of these microorganisms have faster growth rates and are capable of out-competing Euglena absent several controlled growth mechanisms that favor Euglena. These growth mechanisms can include one or more methods such as employment of growth media that favors Euglena, operation at a temperature that favors Euglena, pH levels that favor Euglena, addition of compounds that are toxic to competing organisms other than Euglena, and selective filtration or separation of Euglena. Each of these methods affects the growth rate and the ability of Euglena to convert energy into beta-1,3-glucan. In general, Euglena that are grown in an uncontrolled environment will not display the same beneficial properties of high beta-1,3-glucan concentration, fast growth rates, and efficient production of beta-1,3-glucans that Euglena produced in a more controlled growth environment will display.

In order to achieve cost-efficient large-scale Euglena cultures that efficiently produce beta-1,3-glucan, the organism is generally grown in large aerobic fermentation tanks. Growth media provides a carbon source, a nitrogen source, and other growth nutrients for Euglena growth and beta-1,3-glucan production. The culture media, harvest schedule, and fermentation conditions are carefully controlled to ensure optimal beta-1,3-glucan production. In some embodiments, the production method yields large quantities of Euglena with about 30 wt % to about 70 wt % beta-1,3-glucan, about 30 wt % to about 40 wt % beta-1,3-glucan, about 40 wt % to about 50 wt % beta-1,3-glucan, about 50 wt % to about 60 wt % beta-1,3-glucan, about 60 wt % to about 70 wt % beta-1,3-glucan, about 40 wt % to about 70 wt % beta-1,3-glucan, or about 50 wt % to about 70 wt % beta-1,3-glucan.

Efficient production of beta-1,3-glucan derived from Euglena grown using fermentation reduces the cost of beta-1,3-glucan production in several ways. First, the beta-1,3-glucan produced by Euglena is not contained in the cell wall of the organisms and does not require elaborate and/or expensive fractionation methods or extraction processes, as is required by other organisms known to produce beta glucan. Second, the Euglena organisms are relatively large and may be separated from water relatively quickly by employing a centrifuge, filter, or other separation device. Third, individual Euglena cells are composed of a larger percentage of beta-1,3-glucan (as a percent of total cell mass) in comparison to other organisms, which results in easier recovery of the beta-1,3-glucan. In some embodiments, the Euglena growth is supplemented by light exposure.

### Fermentation Growth of Euglena

The beta-1,3-glucan derived from Euglena useful for the compositions and methods described herein may be produced by growing the Euglena using fermentation. Generally, growth media is provided to the Euglena such that the culture grows heterotrophically. However, it is contemplated that the Euglena can be grown in at least partial exposure to light. The large-scale production of beta-1,3-glucan is substantially more cost effective when the Euglena are heterotrophically fermented rather than grown using photosynthesis, due in part to the large-scale set-up of photosynthetic growth conditions for the algae and the increased cell density obtainable during growth using fermentation.

Exemplary methods of growing Euglena using fermentation are described herein and in U.S. Patent Publication 2013/0303752. These efficient and cost-effective methods allow for the cultivation of Euglena useful for the methods and compositions described herein, including the production of beta-1,3-glucan derived from Euglena.

The Euglena grown using fermentation is cultivated using a growth medium. The growth medium provides nutrients to the growing Euglena culture, including a carbon source, a nitrogen source, and other micronutrients. The growth medium also includes a buffer to maintain the pH of the growth culture. To prevent the growth of unwanted organisms (such as bacteria), the growth medium is sterilized prior to being added to the fermentation tank. The growth medium can be sterilized, for example, by using a filter, steam, autoclaving, or a combination thereof. Optionally, different components of the medium are held in separate storage takes to prevent the formation of a complete growth medium during storage and contamination of the growth medium.

The fermenting Euglena relies on a carbon source present in the growth medium. Example carbon sources include glucose, dextrose, or other sugars; acetate; or ethanol. In some embodiments, the Euglena are grown in a growth medium with a carbon source at about 50 g/L or less, about 40 g/L or less, about 30 g/L or less, about 25 g/L or less, about 20 g/L or less, about 15 g/L or less, about 10 g/L or less, about 5 g/L or less, about 4 g/L or less, about 3 g/L or less, about 2 g/L or less, about 1 g/L or less, about 0.5 g/L or less, or about 0.1 g/L or less. Optionally, the growth medium is supplemented with additional carbon source during the course of growth. For example, the carbon source can be added two or more times to the growth medium, three or more times to the growth medium, or four or more times to the growth medium during the course of Euglena culture growth. The carbon source can be added semi-continuously. The carbon source can also be continuously added to the growth media.

The growth medium useful for growing Euglena by fermentation also includes a nitrogen source, such as ammonium hydroxide, ammonium gas, ammonium sulfate, or glutamate. In some embodiments, the growth medium includes about 0.1 g/L to about 3 g/L nitrogen source, about 0.2 g/L to about 2 g/L nitrogen source, or about 0.5 g/L to about 1 g/L nitrogen source. Preferably, the nitrogen source is ammonium hydroxide.

The growth medium further includes additional nutrients necessary for Euglena culture. For example, the growth medium can include potassium phosphate (such as about 0.25 g/L to about 5 g/L potassium phosphate, about 0.5 g/L to about 4 g/L potassium phosphate, or about 1 g/L to about 3 g/L potassium phosphate), magnesium sulfate (such as about 0.25 g/L to about 5 g/L magnesium sulfate, about 0.5 g/L to about 4 g/L magnesium sulfate, or about 1 g/L to about 3 g/L magnesium sulfate), calcium chloride (such as about 0.005 g/L to about 0.5 g/L calcium chloride, about 0.01 g/L to about 0.4 g/L calcium chloride, or 0.1 g/L to about 0.25 g/L calcium chloride), or a trace metal stock solution comprising micronutrients.

Maintaining the pH of the growth media allows for efficient beta-1,3-glucan production, Euglena cell growth, and helps limit the growth of unwanted bacteria. A pH of about 3 to about 4 is favorable to Euglena, but provides lower than the optimal growth conditions for most bacteria. In some embodiments, the pH of the growth medium is about 2 to about 7, about 2 to about 6, about 3 to about 5, about 3 to about 4, or about 3 to about 3.5. A buffer, for example citrate salt and/or citric acid, can be included in the growth media to maintain the pH of the growth medium in the desired range.

The desired pH of the growth medium may be achieved or maintained in several ways. The pH of the growth medium can be manually monitored and acid or base periodically added manually to reach the desired pH of the growth medium. The pH of the growth medium can alternatively or additionally be measured with a pH sensor connected to an automated control system, and the automated control system controls pumps, hoppers, or other devices that automatically adds acid or base to reach the desired pH of the growth medium that is programmed into the automated control system. In some embodiments, the metabolic processes of the Euglena sufficiently regulate the pH of the growth medium within the desired range.

To provide sufficient oxygen to the Euglena during fermentation, the growth medium can optionally be oxygenated, for example to about 0.5 mg/L to about 4 mg/L oxygen, about 1 mg/L to about 3 mg/L oxygen, or about 2 mg/L oxygen. The cell media can be oxygenated before being added to the fermentation tank or the fermenting Euglena culture can be mixed to facilitate dissolving ambient oxygen into the growth media.

Systems for fermenting Euglena can include one or more bioreactors. The Euglena culture is grown in the bioreactor to a specified cell density or a specified length of time before being the culture is either harvested or used to inoculate a larger bioreactor. Optionally, a portion of the Euglena culture can remain in the bioreactor to inoculate fresh growth media added to the bioreactor. The Euglena grown using fermentation can be grown in a multi-stage process, which may require two or more, three or more, or four or more bioreactors wherein the contents of an earlier bioreactor are transferred to and diluted in a later bioreactor. In another example of fermenting Euglena, the Euglena cell culture is grown using a fed-batch process, wherein fresh growth media or specific media components are continually added to the bioreactor as the Euglena culture grows. A repeated batch process can also be used to ferment the Euglena, wherein the Euglena culture is harvested at regular intervals or continuously harvested and replaced by fresh growth media.

In one example, the Euglena is grown in a single bioreactor, or a fermentation tank. Cell growth media is added to the bioreactor and inoculated with a Euglena culture. The Euglena culture can be, for example, a culture from a different bioreactor or a Euglena colony selected from a growth plate. In some embodiments, the single bioreactor is about 100 liters or larger, about 200 liters or larger, about 300 liters or larger, about 500 liters or larger, about 750 liters or larger, about 1,000 liters or larger, about 5,000 liters or larger, about 10,000 liters or larger, about 15,000 liters or larger, or about 20,000 liters or larger. The Euglena ferment in the bioreactor before being harvested.

The Euglena culture can also grow in a multi-stage fermentation process, wherein multiple bioreactors are used in sequence. In a multi-stage fermentation process, each bioreactor has a larger bioreactor volume than the bioreactor in the preceding bioreactor. A Euglena culture grows in a first to reach a certain cell density. The culture is then used to inoculate the next sequential bioreactor.

Purification of Beta-1,3-Glucan Derived from Euglena Grown Using Fermentation

The beta glucan can be extracted from the Euglena through a liquid/solid separation, a physical separation method, or another method. A substantial portion of the beta-1,3-glucan produced by Euglena is in the form of paramylon. The paramylon is generally present in Euglena in the form of water-insoluble granules of about 0.5 to about 2 microns in size and located within the Euglena cells. Therefore, the beta-1,3-glucan is generally purified by lysing the Euglena cells and isolating the beta-1,3-glucan from the residual biomass. Optionally, the beta-1,3-glucan is purified using methanol. Preferably, the beta-1,3-glucan is purified without the use of chloroform.

The beta-1,3-glucan derived from Euglena is extracted by lysing the cells and isolating the beta-1,3-glucan. The Euglena cells can be lysed using sonication or high pressure homogenization. Optionally, lysing chemicals are included during the lysis step. However, it is possible to lyse the Euglena cells without the addition of lysing chemicals. Exemplary lysing chemicals that could be included during the lysis step include detergents (such as sodium dodecyl sulfate), enzymes, bases (such as sodium hydroxide), or acids (such as acetic acid or hydrochloric acid). After lysing the Euglena cells, the beta-1,3-glucan is isolated using filtration or gravity separation (such as gravity settling or centrifugation). The isolated beta-1,3-glucan can then be washed, for example with an aqueous solution or an ethanol, to obtain higher purity.

After purification of the beta-1,3-glucan derived from Euglena, additional processing steps can modify the purified beta-1,3-glucan. Modified beta-1,3-glucan displays increased binding affinity to immune system receptors, such as Dectin-1, a protein that has been identified as a beta glucan receptor. For example, sulfated polysaccharides have been demonstrated to display anti-HIV activity (e.g., U.S. Pat. No. 5,861,383). In one exemplary method of preparing a sulfated beta-1,3-glucan, the purified beta-1,3-glucan is dissolved in dimethyl sulfoxide and combined with a mixture of dry pyridine and chlorosulfonic acid. The mixture is then heated and the supernatant is decanted. Subsequently, distilled water or methanol is added to the supernatant in order to precipitate pyridinium beta-1,3-glucan sulfate, which can then be collected by filtration. Alternatively, sodium chloride is added to the supernatant and the pH is raised to 9, allowing the sodium beta-1,3-glucan sulfate to precipitate in an acetone solution (see Sakagami et al., In vivo 3:243-248 (1989)).

Beta-1,3-glucan derived from Euglena can also be modified to be cationic. Cationic beta glucan can be more biologically active as an immunomodulator, as it has increased binding affinity with beta glucan receptors such as Dectin-1 and complement receptor 3 (see Sakagami et al., Antiviral Research, 21:1-14 (1993)). Beta-1,3-glucan derived from Euglena can be modified with dimethylethanolamine (DMAE) to produce the cationic beta-1,3-glucan. One exemplary method of producing DMAE beta-1,3-glucan comprises dissolving the beta-1,3-glucan derived from Euglena in a base solution (such as a solution comprising NaOH), and adding a DMAE-chloride (either as a solution or dried powder). The resulting reaction produces DMAE beta-1,3-glucan.

After purification beta-1,3-glucan derived from Euglena can be solubilized using a solubilizing agent as described herein. Various agents may be used to facilitate solubilization such as heat, bases, chaotropic agents, and detergents. In some variations, a base is used to solubilize the beta-1,3-glucan derived from Euglena. Suitable bases include sodium hydroxide, potassium hydroxide, calcium hydroxide, magnesium hydroxide, sodium carbonate, and ammonium. In some variations, a strong base is used to solubilize the beta-1,3-glucan. In other variations, a weak base is used to solubilize the beta-1,3-glucan.

Various amounts of base can be used to solubilize the beta-1,3-glucan. In some variations, an amount of base effective to solubilize the beta-1,3-glucan is used. For example, 0.2M to 10M of base can be used. In some variations, beta-1,3-glucan is solubilized in a solution with 2M, 1.9M, 1.8M, 1.7M, 1.6M, 1.5M, 1.4M, 1.3M, 1.2M, 1.1M, 1M, 0.9M, 0.8M, 0.7M, 0.6M, 0.5M, 0.4M, 0.3M, or 0.2M, sodium hydroxide. In variations when a base is used to solubilize the beta-1,3-glucan, an acid can be added to neutralize the pH of the solution after the beta-1,3-glucan is solubilized with base. For example, following solubilization with base, the pH of a solution comprising beta-1,3-glucan can be adjusted using HCl.

In some variations, beta-1,3-glucan may be solubilized using a chemical other than a base. For example, in some of these variations, beta-1,3-glucan may be solubilized by incubating with a chaotropic agent such as urea or guanidine. In some of these variations, the beta-1,3-glucan is solubilized by incubation with 8M urea or 6M guanidine hydrochloride. In other variations, beta-1,3-glucan is solubilized by incubating with dimethylsulfoxide. In yet other variations, a detergent such as a zwitterionic or non-ionic detergent is used to facilitate solubilization. Exemplary detergents include Tween, octyl-glucose, CHAPS and CHAPSO.

Solubilization can be performed at a range of temperatures. For example solubilization may be performed at temperatures between 4° C. and 200° C. In some variations, solubilization is performed by incubation at room temperature. In other variations, solubilization is performed by incubation at 30° C., 40° C., 50° C., 60° C., 70° C., 80° C., 90° C., or 100° C.

Solubilization can be performed at a range of pressures. For example, solubilization may be performed at pressures between 0.5 atm and 100 atm. In some variations, solubilization is performed by incubation at ambient pressure (1 atm). In other variations, solubilization is performed by incubation at 1.5 atm, 2 atm, 3 atm, 4 atm, 5 atm, 10 atm, 25 atm, 50 atm, 75 atm, or 100 atm. In some variations, solubilization is performed by incubation in an autoclave.

In some variations, beta-1,3-glucan can be solubilized by incubating with a solubilizing agent such as a base, chaotropic agent, solvent, or detergent for varying amounts of time. For example, in some variations, the beta-1,3-glucan can be solubilized by incubating with a solubilizing agent for between 1 minutes and 240 minutes. In some of these variations, the beta-1,3-glucan is solubilized by incubating with a solubilizing agent for about 30 minutes, about 60 minutes, about 90 minutes, about 120 minutes, about 150 minutes, about 180 minutes, about 210 minutes or about 240 minutes.

In some variations, it may be beneficial to alter the beta glucan chain length using enzymes, catalysis, heat, sonication, or combinations thereof as described herein. Additionally, it can be beneficial to start with a highly pure linear source of beta-1,3-glucan, such as beta-1,3-glucan derived from Euglena gracilis, in order to achieve a desired range of optimal target chain lengths.

In another aspect of the present invention, the solubilized beta-1,3-glucan is administered to a human or animal. In at least one embodiment, the solubilized beta-1,3-glucan is combined into animal feed. When combined into animal feed, the Euglena derived beta glucan may be combined at a range of dosing levels, but generally this level can be between 1:10,000 and 1:500 by dry weight. Specific ingredient combinations may differ between organisms, life stages, and the desired outcomes. Additionally, Euglena derived beta glucans can be combined with other immune-stimulating ingredients in order to provide the maximum immune stimulation benefits. Example ingredient combinations are listed below for poultry, swine, and canine applications. Algae or protist-derived may be combined with any combination of (but not limited to) these ingredients in order to make an animal feed product.

There are many animal feed ingredients that may also benefit from combination with solubilized beta-1,3-glucan. Common animal feed components, for example, can include one or more of the following ingredients: corn meal, dehulled soybean meal, wheat middlings, limestone, monocalcium-dicalcium phosphate, salt, manganous oxide, manganese sulfate, zinc oxide, ferrous sulfate, copper sulfate, cobalt carbonate, calcium iodate, sodium selenite, vitamin A, vitamin D, vitamin E, Menadioane sodium bisulfate complex (source of vitamin K complex), riboflavin supplement, niacin supplement, calcium pantothenate, vitamin B12, d-biotin, thiamine mononitrate, pyridoxine hydrochloride, folic acid, methionine, soybean oil, mineral oil, amino acids, Chicken, calcium, phosphorus, chrondrotin, glucosamine, Omega 3 & Omega 6, beet pulp, DHA (from fish oil), beta carotene, fish meal, Vitamin blend, alpha-linlenic acid, amino acids, arachidonic acid, ascorbic acid, beef, biotin, brewers yeast (dried), calcium carbonate, cellulose, chelated minerals, chondroitin sulfate, cobalt, copper, corn meal, corn oil, dicalcium phosphate, DL-methionine, docosahexaenoic acid, dried egg product, durum flour, ethoxyquin, fat, carbohydrate, ferrous sulfate, fiber, fish meal, fish oil, flax meal, folic acid, fructooligosaccharides, gelatin, glucosamine hydrochloride, glycerin, ground barley, ground corn, ground sorghum, guar gum, inositol, iodine, iron, Kangaroo, lamb, 1-carnitine, linoleic acid, lutein, magnesium, magnesium oxide, manganese, marigold extract, mannanoligosaccharides, minerals, mixed tocopherols, monosodium phosphate, niacin, marigold extract, blueberries, dried kelp, phosphorus, potassium, potassium chloride, potassium iodide, potassium sorbate, protein, pyridoxine hydrochloride, riboflavin, rice, rice flour, rosemary, rosemary extract, tapioca starch, taurine, thiamine mononitrate, titanium dioxide, vitamin A, vitamin B-1, vitamin B12, vitamin B-2, vitamin B-6, vitamin C, vitamin D3, vitamin E, vitamin K, water, wheat, wheat glutens, xanthan gum, zinc, zinc oxide, zinc sulfate, any of the ingredients presently listed by the Association of American Feed Control Officials, and combinations thereof.

The following ingredients are related to enhanced immune system performance and can be combined with Euglena derived beta glucans or meal in order to achieve the effects of enhanced immune system activity: vitamin C, alfalfa, flax seed, parsley, cranberries, spirulina, chlorella, vitamin A, vitamin E, copper, zinc, chromium, iron, arginine, alklyglcerol, coenzyme Q10, dimethglycine, phytonutrients, beta carotene, essential oils, fish oils, spices and their derivatives, and combinations thereof.

The ingredients above may be used in various applications and for feeding various organisms. For example, the ingredients listed herein as animal feed components may also be combined with algae or protist-derived beta glucans for dog, cat, poultry, aquaculture and other feed applications. In addition to the immune stimulation benefits of Euglena derived beta glucans, the additional algae biomass may be incorporated. In particular, Euglena gracilis or another species may be grown such that relatively high concentrations of valuable DHA, Omega 3 fatty acid, Omega 6 fatty acid, and tocopherols are also added to the feed composition.

Although beta glucan can be beneficial when included with one or more feed ingredients, there may be certain synergistic effects when beta glucan is fed in combination with one or more additional substances. For example, beta glucan may be fed in combination with probiotics such as Bacillus licheniformis or Bacillus subtilis to provide a synergistic effect. In this embodiment the up-regulation of the immune system may help the body to naturally fight invasive pathogens while the probiotics maintain a healthy intestinal flora that are more stable to overturn. Beta glucan that is fed in combination with other types of non-digestible fibers (e.g., prebiotics) may also exhibit a synergistic effect. Examples of prebiotics that may be beneficially combined with beta glucan include but are not limited to fructooligosaccharides (FOS), lactulose and mannan oligosaccharides (MOS). Prebiotics combined with beta glucan may be derived from yeast, micro-algae, grains, kelp, other terrestrial plants, and other sources. Other substances that may be beneficial in combination with beta glucan include vitamin C, vitamin E (specifically RRR alpha tocopherol), carotenoids (Astaxanthin, beta-carotene, lutein, zeaxanthin), DHA or EPA fatty acids, trace metals (iron, magnesium, lithium, zinc, copper, chromium, nickel, cobalt, vanadium, molybdenum, manganese, selenium, iodine), halquinol, ME Detoxizyme, vitamin D3, ascorbic acid, and dietary minerals (calcium, phosphorus, potassium, sulfur, sodium, chlorine, magnesium, boron, chromium). Beta glucan may also be fed in combination with other enzymes, which may improve the bioavailability or digestibility of one or more nutrient sources in the feed. In some cases, beta glucanase may be provided as an enzyme in the feed to cleave the beta glucan into smaller, more digestible fragments or to release the metal from a metal beta glucan complex. In some embodiments, one or more of these additional substances can be included in the residual algae meal, which may be cultivated with the intent of increasing the concentration of the synergistic substances.

Further ingredients can be combined with beta glucan and the various beta glucan compositions described herein. These include an additional immune modulating, stress reducing, or other stimulant ingredient selected from the group consisting of alpha tocopherol, cholecalciferol, zinc, chromium, selenium, arginine, ascorbic acid, alklyglcerol, caffeine, kava kava, curcuma longa, spirulina, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene, and combinations thereof.

### Examples

### Example 1: Comparison of Particulate and Soluble Beta-1,3-Glucan Induced Response by Macrophages In Vitro

Purified beta-1,3-glucan derived from Euglena was weighed and suspended in pyrogen-free water. The resulting suspension contained 20 mg/mL (2 weight %) of beta-1,3-glucan derived from Euglena. 2M NaOH was added to a final concentration of 1M and the samples were incubated at room temperature for two hours with intermittent mixing to solubilize the beta-1,3-glucan. 10× phosphate buffered saline (PBS) was then added to the solution. The pH was neutralized using 1N HCl and the volume was adjusted to a final concentration of 4 mg/ml beta-1,3-glucan in 1×PBS.

Peritoneal macrophages from naïve mice were cultured (1×106 cells/ml) in the presence of different amounts of particulate or soluble beta-1,3-glucan for 48 hours in triplicate. Supernatants were tested for secreted cytokines IL-6, IL-1β, IL-10, IL-1α, TNFα, IL-12, MCP-1, IP-10, Rantes, and MIP-1α by multiplex assay. Pro-inflammatory agent, lipopolysaccharide (LPS) was used as a positive control. Flow cytometry was also used to detect the activation markers CD86 and MHC II using fluorochrome labelled antibodies to CD86 or MHCII.

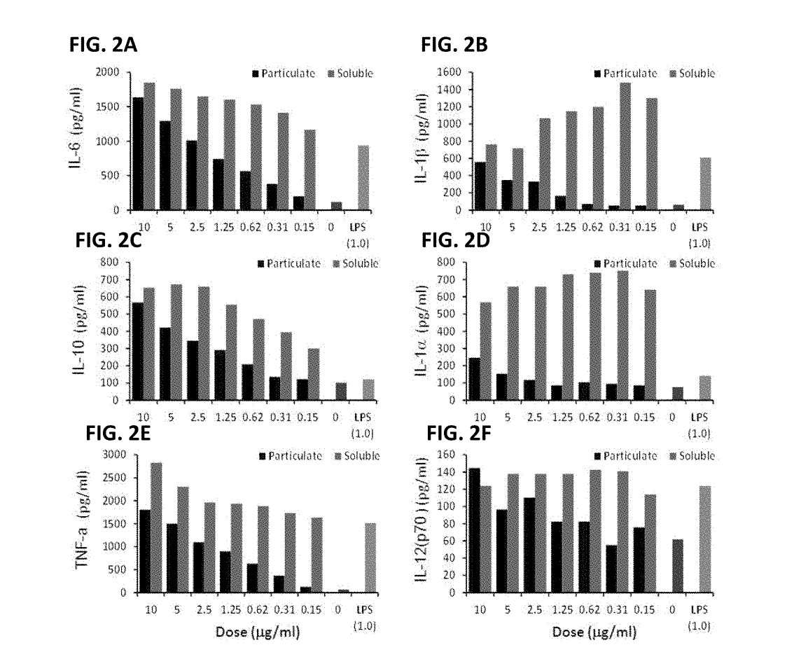

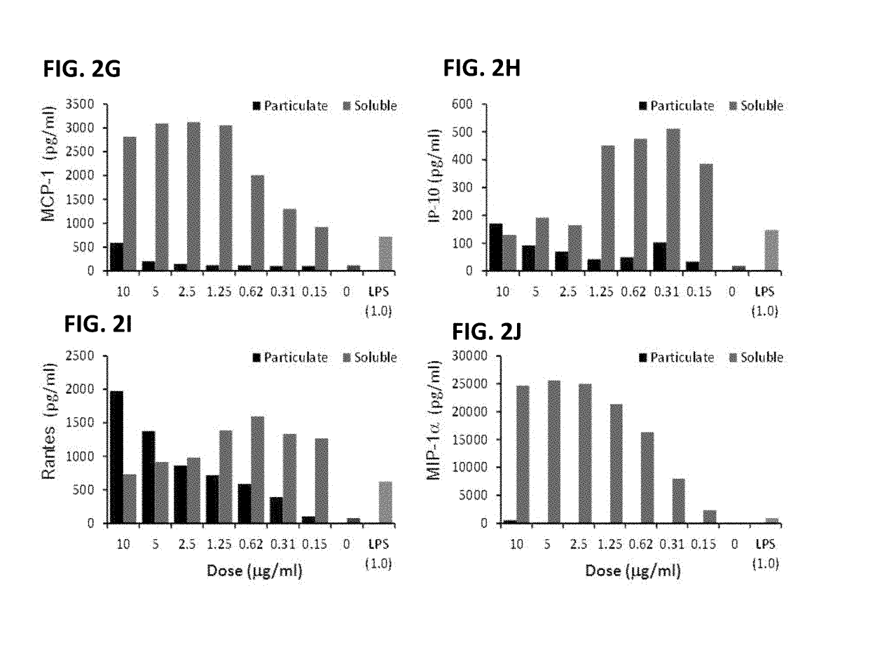

FIGS. 2A-2J summarize the results of the in vitro assay for cytokine production by peritoneal macrophages cultured in the presence of varying amounts of solubilized or particulate Euglena-derived beta-1,3-glucan. The mean concentration of cytokines across three replicates is plotted. As shown in FIGS. 2A-2J, solubilized Euglena-derived beta-1,3-glucan induced release of cytokines from macrophages much more efficiently than particulate beta-1,3-glucan, even at low concentrations. High levels of many of the cytokines tested were produced when cells were treated with the lowest dose of soluble beta-1,3-glucan tested (0.15 μgimp, whereas similar cytokine production levels were not observed even at the highest doses of particulate beta glucan (10 μgimp that was tested, for many of the cytokines (FIGS. 2B, 2D, 2E, 2G, 2H, and 2J).

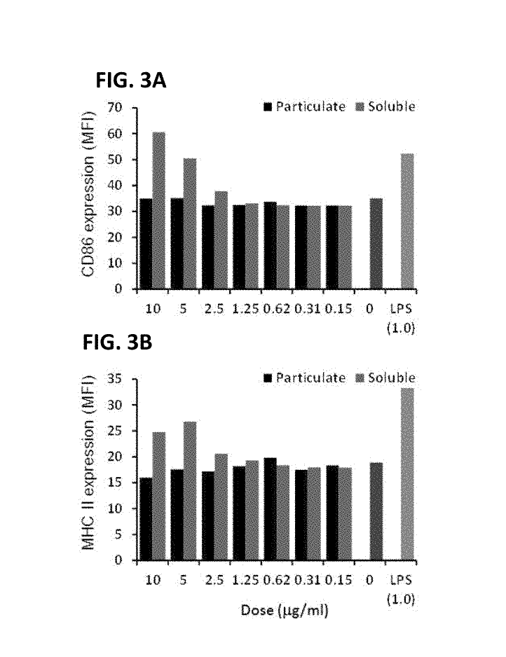

FIGS. 3A-3B show the results of flow cytometric analysis of macrophages treated with soluble and particulate beta-1,3-glucan. Treatment of macrophages with 2.5, 5, and 10 μg of solubilized beta-1,3-glucan led to progressively increased expression of CD86 to levels above those observed in the LPS-treated control (FIG. 3A). Similarly, treatment of cells with 2.5, 5, and 10 μg of soluble beta-1,3-glucan resulted in increased MHC II expression compared to the untreated control, whereas no similar increase was observed for cells treated with particulate beta-1,3-glucan (FIG. 3B).

This study demonstrates that solubilized beta-1,3-glucan derived from Euglena is able to stimulate professional antigen-presenting cells such as macrophages more efficiently than the particulate form. This suggests that solubilized beta-1,3-glucan may be an effective treatment for enhancing an immune response in an individual.

### Example 2: Comparison of Particulate and Soluble Beta-1,3-Glucan Induced Response by Dendritic Cells In Vitro

Purified beta-1,3-glucan derived from Euglena was weighed and suspended in pyrogen-free water. The resulting suspension contained 20 mg/mL (2 weight %) of beta-1,3-glucan derived from Euglena. 2M NaOH was added to a final concentration of 1M and the samples were incubated at room temperature for two hours with intermittent mixing to solubilize the beta-1,3-glucan. 10× phosphate buffered saline (PBS) was then added to the solution. The pH was neutralized using 1N HCl and the volume was adjusted to a final concentration of 4 mg/ml beta-1,3-glucan in 1×PBS.

Bone marrow derived murine dendritic cells (DCs) were cultured (1×106 cells/nil) in the presence of different amounts of particulate or solubilize beta-1,3-glucan for 48H in triplicate. Supernatants were tested for secreted cytokines IL-6, IL-1β, IL-10, IL-1α, TNFα, IL-12, MCP-1, IP-10, Rantes, and MIP-1α by multiplex assay. Pro-inflammatory agent, lipopolysaccharide (LPS) was used as a positive control. Flow cytometry was also used to detect the activation markers CD86 and MHC II using fluorochrome labelled antibodies to CD86 or MHCII.

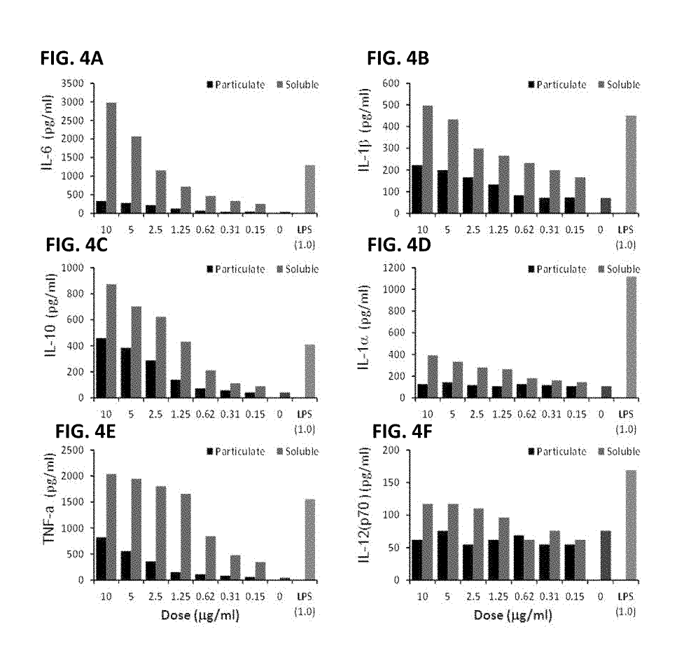

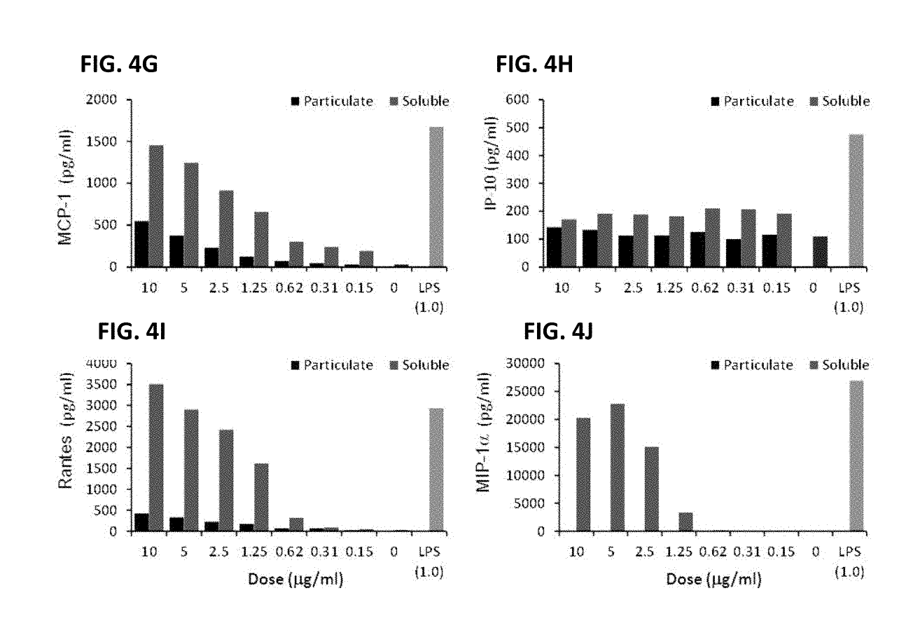

FIGS. 4A-4J show the results of the in vitro assay for cytokine production by dendritic cells cultured in the presence of varying amounts of solubilized or particulate Euglena-derived beta-1,3-glucan. The mean concentration of cytokines across three replicates is plotted. As shown in FIGS. 4A-4J, solubilized Euglena-derived beta-1,3-glucan induced release of cytokines from dendritic cells much more efficiently than particulate beta-1,3-glucan, even at low concentrations.

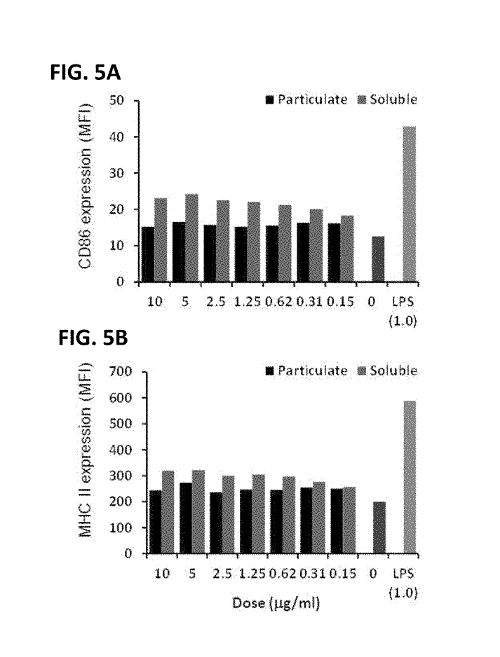

FIGS. 5A-5B show the results of flow cytometric analysis of expression of CD86 and MHCII in dendritic cells treated with soluble or particulate beta-1,3-glucan. Expression levels of CD86 (FIG. 5A) and MHCII (FIG. 5B) were increased in cells treated with soluble beta glucan, compared to cells treated with particulate beta glucan or untreated control.

This study demonstrates that solubilized beta-1,3-glucan derived from Euglena is more efficient than the particulate form of beta-1,3-glucan at activating the professional antigen-presenting cell type of dendritic cells. This study further demonstrates that solubilized beta-1,3-glucan may be an effective treatment for enhancing immune function in an individual.

### Example 3: Soluble Beta-1,3-Glucan Induced Immune Response In Vivo

Purified beta-1,3-glucan derived from Euglena was weighed and suspended in pyrogen-free water. The resulting suspension contained 20 mg/mL (2 weight %) of beta-1,3-glucan derived from Euglena. 2M NaOH was added to a final concentration of 1M and the samples were incubated at room temperature for two hours with intermittent mixing to solubilize the beta-1,3-glucan. 10× phosphate buffered saline (PBS) was then added to the solution. The pH was neutralized using 1N HCl and the volume was adjusted to a final concentration of 4 mg/ml beta glucan in 1×PBS.

Five mice per group were treated by daily oral gavage with PBS (control), 500 μg particulate beta-1,3-glucan (pAG500), 500 μg solubilized beta-1,3-glucan (sAG500), 125 μg solubilized beta-1,3-glucan (sAG125), or 50 μg solubilized beta-1,3-glucan (sAG50) for seven days. Mice were euthanized on day 8 and ileum tissues were collected. After cleansing, single cell suspensions were made from ileum of individual mice by collagenase digestion. The immune cell fraction was enriched using the MACS enrichment protocol (Miltenyl Biotech). Immune cells were cultured (1×106 cells/nil) over night and then supernatants were tested for cytokines by multiplex assays in duplicate.

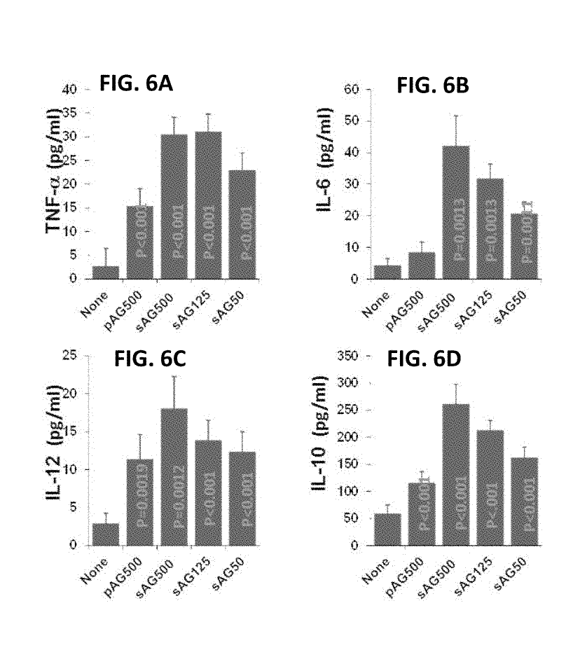

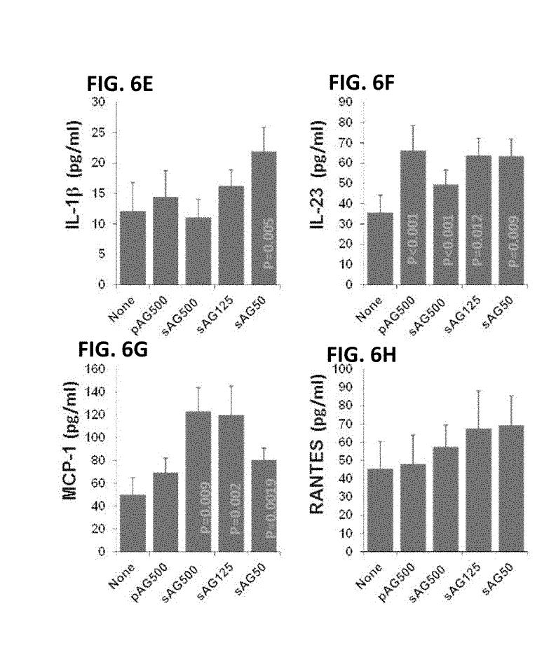

FIGS. 6A-6H show cytokine production of immune cells derived from mice treated with particulate or soluble beta-1,3-glucan. Treatment with 500 μg particulate beta-1,3-glucan (pAG500) increased production of TNF□ (FIG. 6A), IL-12 (FIG. 6C), IL-10 (FIG. 6D), and IL-23 (FIG. 6F) compared to the untreated control. Cytokine production was further increased by treatment with as little as 50 μg solubilized beta-1,3-glucan (FIGS. 6A-6F).

These results suggests that beta-1,3-glucan derived from Euglena and solubilized by base may be more bioactive and may possess stronger immunomodulatory activity in vivo compared with beta-1,3-glucan provided in a particulate form.

### Example 4: Use of Detergent to Facilitate Solubilization of Beta-1,3-Glucan

Purified beta-1,3-glucan derived from Euglena is weighed and suspended in phosphate buffered saline (PBS). The resulting suspension contains 20 mg/mL (2 weight %) of beta-1,3-glucan derived from Euglena. CHAPS is added to facilitate solubilization in the PBS to a final concentration of 5-10 mM. The samples are incubated at room temperature for two hours with intermittent mixing to solubilize the beta-1,3-glucan. The solubilized beta-1,3-glucan is diluted to a final concentration of 4 mg/ml in 1×PBS.

Bone marrow derived murine dendritic cells (DCs) and murine macrophages are cultured (1×106 cells/ml) in the presence of different amounts of particulate or detergent-solubilized beta-1,3-glucan for 48 hours in triplicate. Cytokine levels are detected. MHC II and CD86 levels are detected using ELISA.

Cytokine levels are increased upon treatment with as little as 0.15 μg/ml of detergent-solubilized the beta-1,3-glucan. MHCII and CD86 protein levels are also increased upon treatment with as little as 0.15 μg/ml of detergent-solubilized the beta-1,3-glucan.

These results demonstrate that the use of a detergent to facilitate the solubilization of beta-1,3-glucan results in a more potent immunomodulatory effect than particulate beta-1,3-glucan.

### Example 5: Use of DMSO to Facilitate Solubilization of Beta-1,3-Glucan

Purified beta-1,3-glucan derived from Euglena is weighed and suspended in phosphate buffered saline (PBS). The resulting suspension contains 20 mg/mL (2 weight %) of beta-1,3-glucan derived from Euglena. Dimethyl sulfoxide (DMSO) is added to facilitate the solubilization in the PBS to a final concentration of 10-50%. The samples are incubated at room temperature for two hours with intermittent mixing to solubilize the beta-1,3-glucan. The solubilized beta-1,3-glucan is diluted to a final concentration of 4 mg/ml in 1×PBS and a final DMSO concentration of less than 1%.

Bone marrow derived murine dendritic cells (DCs) and murine macrophages are cultured (1×106 cells/nil) in the presence of different amounts of particulate or DMSO-solubilized beta-1,3-glucan for 48 hours in triplicate. Cytokine levels are detected. MHC II and CD86 levels are detected using ELISA.

Cytokine levels are increased upon treatment with as little as 0.15 μg/ml of DMSO-solubilized the beta-1,3-glucan. MHCII and CD86 protein levels are also increased upon treatment with as little as 0.15 μg/ml of DMSO-solubilized the beta-1,3-glucan.

These results demonstrate that DMSO-solubilized beta-1,3-glucan has a more potent immunomodulatory effect than particulate beta-1,3-glucan.

### Example 6: Use of Heat Treatment to Facilitate Solubilization of Beta-1,3-Glucan

Purified beta-1,3-glucan derived from Euglena is weighed and suspended in phosphate buffered saline (PBS). The resulting suspension contains 20 mg/mL (2 weight %) of beta-1,3-glucan derived from Euglena. Samples are heated to 90-120° C. for 2 hours to facilitate solubilization in the PBS. The solubilized beta-1,3-glucan is diluted to a final concentration of 4 mg/ml in 1×PBS.

Bone marrow derived murine dendritic cells (DCs) and murine macrophages are cultured (1×106 cells/ml) in the presence of different amounts of particulate or heat-solubilized beta-1,3-glucan for 48 hours in triplicate. Cytokine levels are detected. MHC II and CD86 levels are detected using ELISA.

Cytokine levels are increased upon treatment with as little as 0.15 μg/ml of heat-assisted solubilized beta-1,3-glucan. MHCII and CD86 protein levels are also increased upon treatment with as little as 0.15 μg/ml of heat-solubilized the beta-1,3-glucan.

These results demonstrate that heat-solubilized beta-1,3-glucan has a more potent immunomodulatory effect than particulate beta-1,3-glucan.

The above description includes several numerical ranges in the text and Figs. The numerical ranges support any range or value within the disclosed numerical ranges even though a precise range limitation is not stated verbatim in the specification because embodiments of the invention can be practiced throughout the disclosed numerical ranges.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. The entire disclosure of the patents and publications referred to in this application are hereby incorporated herein by reference. Finally, the invention can be construed according to the claims and their equivalents.

1. A method of enhancing the immune function in an individual in need thereof comprising administering to the individual an effective amount of a composition comprising solubilized Euglena-derived beta-1,3-glucan.

2. The method of claim 1, wherein the Euglena-derived beta-1,3-glucan is solubilized by a base.

3. The method of claim 1, wherein the effective amount of the composition is between 0.01 mg beta-1,3-glucan/kg body weight and 100 mg beta-1,3-glucan/kg body weight.

4. The method of claim 1, wherein the solubilized beta-1,3-glucan is more bioactive than a particulate form of beta-1,3-glucan derived from Euglena.

5. The method of claim 1, wherein administration of the composition modulates an autoimmune response, blood sugar level, cholesterol level, an infection, or inflammation.

6. The method of claim 5, wherein the inflammation is associated with allergies or intestinal inflammation.

7. The method of claim 5, wherein the autoimmune response is associated with diabetes.

8. The method of claim 5, wherein the infection is a bacterial, fungal, or viral infection.

9. The method of claim 1, wherein the Euglena is heterotrophically grown.

10. The method of claim 1, wherein the beta-1,3-glucan comprises paramylon.

11. The method of claim 1, wherein the beta-1,3-glucan does not contain beta-1,6-glycosidic bonds.

12. The method of claim 1, wherein the composition is administered daily as a single dose.

13. The method of claim 1, wherein the composition is administered as multiple separate doses in a single day.

14. The method of claim 1, wherein the composition further comprises an additional component selected from the group consisting of alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene, and combinations thereof.

15. The method of claim 1, wherein the composition is administered orally.

16. The method of claim 15, wherein the composition is added to drinking water.

17. The method of claim 1, wherein the composition is administered intravenously.

18. The method of claim 1, wherein the composition is administered topically.

19. The method of claim 1, wherein the composition has a pH greater than 7.

20. The method of claim 1, wherein the composition has a pH less than 7.

21. The method of claim 1, wherein the composition has a pH of approximately 7.

22. The method of claim 1, wherein the composition is administered with one or more components selected from the group consisting of alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene, and combinations thereof.

23. A bioactive composition for enhancing immune function in an individual comprising solubilized Euglena-derived beta-1,3-glucan, wherein the beta-1,3-glucan is present in an amount from 1 ppm to 10 ppm.

24. The composition of claim 23, wherein the Euglena-derived beta-1,3-glucan is solubilized in a solution with the introduction of a base.

25. The composition of claim 23, wherein the Euglena is heterotrophically grown.

26. The composition of claim 23, wherein the beta-1,3-glucan consists essentially of unbranched beta-1,3-glucan.

27. The composition of claim 23, wherein the beta-1,3-glucan does not contain beta-1,6-glycosidic bonds.

28. The composition of claim 23, wherein the composition is a liquid composition.

29. The composition of claim 23, wherein the composition is a gel composition.

30. The composition of claim 23, wherein the composition further comprises an additional component selected from the group consisting of alpha tocopherol, cholecalciferol, zinc, chromium, selenium, arginine, ascorbic acid, alklyglcerol, caffeine, kava kava, curcuma longa, Spirulina, Chlorella, calcium D-glucarate, coenzyme Q10, peptides, dimethglycine, docosahexaenoic acid, ecosapentaenoic acid, alpha-lineolenic acid, astaxanthin, beta carotene, lutein, lactobacillus probiotics, bifidobacterium probiotics, mannoliggosaccharide, fructooliggosacharides, Astragalus, Echinacea, Esberitox, garlic, glutathione, kelp, L-arginine, L-ornithine, lecithin granules, extracts from maiitake, reishi or shiitake mushrooms, manganese, quercetin, bromelain, Olive Leaf, Sambucus, Umcka, panthothenic acid, quercetin, alpha lipoic acid, essential oils, fish oils, spices and their derivatives, pterostilbene, and combinations thereof.

31. The composition of claim 23, wherein the composition further comprises a metal.

32. The composition of claim 31, wherein the metal is selected from the group consisting of iron, magnesium, lithium, zinc, copper, chromium, nickel, cobalt, vanadium, molybdenum, manganese, selenium, and combinations thereof.

33. The composition of claim 31, wherein the beta-1,3-glucan and the metal form a complex.

34. A kit for enhancing the immune function in an individual in need thereof comprising the composition of claim 23 and instructions for use.
